(19) United States
(12) Patent Application Publication
(10) Pub. No.: US 2024/0090922 A1
(43) Pub. Date: Mar. 21, 2024

(54) Miniature Angle-View Endoscope with Image Orientation Correction

(71) Applicant: IntraVu Inc., Redwood City, CA (US)

(72) Inventors: TheofiIos Kotseroglou, Hillsborough, CA (US); Stephanos Papademetriou, Woodside, CA (US); Stephen A. Brown, Oxford Station (CA); Ulrich R. Haug, Campbell, CA (US); Efthalia K. Kotseroglou, Hillsborough, CA (US)

(21) Appl. No.: 17/737,902

(22) Filed: May 5, 2022

Related U.S. Application Data
(60) Provisional application No. 63/184,700, filed on May 5, 2021.

(51) Int. Cl. A61B 17/34; A61B 1/00; A61B 90/00; H04N 5/232

(52) U.S. Cl. CPC A61B 17/3478; A61B 1/00183; A61B 90/361; H04N 5/23296; A61B 2017/3454

(57) ABSTRACT

A rotatable chip-on-tip endoscope is configured to be operated with a single hand, with the palm holding the body of the device and one or two fingers operating a distal rotatable end of the handle that carries the needle. The needle is small in diameter, preferably less than 2.5 mm at its largest end. As the tip is rotated to view more widely in a patient's joint, for example, video processing electronics correct rotational orientation in real time to present a consistent moving image on a monitor.

This application claims benefit of provisional application No. 63/184,700, filed May 5, 2021.

### BACKGROUND OF THE INVENTION

The invention concerns surgical endoscopes and use of endoscopes. In particular the invention is directed to improvements in small-diameter rotatable chip-on-tip endoscopes.

During surgery, mismatches in the spatial orientation between the visual display space, e.g. the monitor of the operating room, and the physical environment, e.g. inside a patient joint, can lead to a reduced surgical performance. Such mismatches occur in use of side-viewing endoscopes.

Hence, in order to assist surgeons interpreting and reading images from video-endoscopy, automated or semi-automated image rectification or re-orientation according to a pre-defined main axis is desirable.

Furthermore, it is common practice in surgeries to use a side-viewing endoscope, in order to visualize not only forward looking areas, but also specific items that may be located on either side of the main, forward-located image. Sometimes it is necessary to locate a defect that exists behind an obstacle on the side of the endoscope tip, which is only accessible with a side viewing imaging system. That also alleviates the need to use flexible or steerable scopes, which have their own issues. Fully viewing a region with a side-viewing scope involves rotation of the scope, but if the sensor (camera) is included in rotation, changes will occur in rotational orientation of the image on the monitor.

Variable viewing directionality for side-viewing endoscopes has been achieved conventionally mainly via an optical element that is rotated with respect to the imaging sensor or camera, which is fixed. This does not result in a rotating image on a monitor.

A side-viewing endoscope typically uses a camera mechanically connected (outside the body) to a lens system necessary for imaging objects from inside the body onto the camera, where the side-viewing lens system can be rotated about a longitudinal axis of the scope. The lens system itself has an optical axis at its distal end that is set at a specific angle with respect to the axis of the forward pointing scope, i.e. to the longitudinal axis of the scope. In most cases it is necessary to minimize the size or diameter of the scope in order to access small cavities in the body or generate minimal friction with the body and minimal injury.

The camera/imaging sensor is held firm and stable with respect to the patient or to a general frame of reference in the room. That means that even if the side-viewing scope is rotated via an optical element to access viewing directions other than the forward direction, it is not necessary to modify the image collected in a digital way in order to view the image on a video monitor in the specific orientation desired: If the sensor is situated with its horizontal dimension parallel to the horizon, then rotation of the lens in current side-viewing scopes does not affect the orientation of the image on the video monitor, i.e. “up” on the video monitor is also “up” with respect to the patient or body part under inspection.

A specific case of endoscopes are “chip-on-tip” endoscopes where the camera imaging sensor is fixedly attached to the lens and both are located at the distal end of the endoscope. See, for example, U.S. Pat. No. 10,463,399. The chip-on-tip endoscopes can have a flexible body from a proximal end to the distal camera-lens system or can be a stiff construct that connects to a handle at the proximal end. In the latter case the handle and camera and lens have all been integrated and rigidly attached to each other.

In all current background cases of chip on tip architectures with a fixed small lens system there are two options: One is a stiff “needle” endoscope wherein the operator's hand can manipulate the tip of the endoscope (by manipulating the handle it is rigidly attached to) and thus its aim without having to consider any motion, rotation, twist of the base or body of the endoscope. Usually the stiff chip-on-tip endoscopes are short in length on the order of one foot or shorter. The other option is a flexible endoscope where the chip-on-tip is at the tip of the scope and a flexible member of possibly longer length, even up to several meters, separates the handle and the tip. This would be also called a boroscope in some others non-health related applications.

In these current background devices, if a rotation of the tip is needed in order to view an otherwise non-accessible location of a target area, there is in many cases the need to correct the orientation of the image in the video monitor and retain that image always at a specific rotational orientation with respect to specific fiducials of the target, or relative to the horizon. If the target for example is a patient's knee, there is a need to keep the image in the video monitor such that the horizon is preserved. To achieve that functionality some chip-on-tip systems have some form of rotation sensor and electronic correction of image orientation.

First, a small sensor can be incorporated with a larger and longer variable lens in front of the sensor and the variable lens can rotate. This is not a true chip-on-tip, since in this construct the lens extends far past the imaging sensor and extends the length of the scope so that the lens can be rotated with respect to the sensor. These devices resemble miniaturized bulkier side-viewing stiff scopes instead of chip-on-tip endoscopes. Additionally, the more complex lens system distal to the sensor increases the overall diameter of the scope far beyond the footprint of the underlying sensor and results in a much larger-diameter construct. Furthermore, such devices are more complex and expensive and defeat a primary purpose of disposability in chip-on-tip endoscopes.

Second, chip-on-tip scopes can be integrated with electronic devices (i.e. electronic gyro at the tip) that will transmit the rotation information of the tip to a computer which in turn will use this information to correct for the angle of rotation and project the image in the video screen with its corrected upright orientation.

These devices are complex, and true orientation or at the very least reliable orientation information may not be known unless an accelerometer and a magnetometer are included on the tip. Electronic component signals can drift over time, especially in devices stored or transported before use or the next use, creating a need for a “reset” or calibration before such use. Also, the devices are expensive for specifically disposable endoscopes and arthroscopes. Furthermore, the addition of electronic sensors near or around the distal tip of the scope will increase the diameter of the scope, an undesirable effect especially in medical applications where the goal is to devise the smallest possible construct for minimal tissue damage.

### SUMMARY OF THE INVENTION

According to the current invention, a chip-on-tip endoscope consists of an imaging lens and imaging sensor at the distal tip of a small diameter rigid shaft. The optical axis of the imaging lens can be along the axis of the shaft (zero-degree forward looking scope) or at some acute angle (side-viewing scope) with respect to the axis of the shaft (for example 30-degree side-viewing scope). The proximal end of the small diameter shaft is attached to a handle which allows the user to hold the instrument and manipulate it while inside the body.

The handle is constructed so that its distal portion (that is rigidly attached to the proximal end of the shaft) can rotate relative to the base portion of the handle. This way, in the case of a side-viewing scope, new information can be revealed to the user with rotation. A rotation-sensing transducer is located in the handle proximal to the small diameter shaft and is mechanically communicating with the rotating portion of the handle. This way any rotation of the shaft (and thus the imaging sensor) can be sensed and communicated to an imaging processing unit (that is either wired or wirelessly connected to the handle) for further image manipulation and orientation correction.

Illumination for imaging is provided by either optical fibers running through the length of the shaft, receiving light from an LED source within the handle, or by a smaller LED or series of smaller LEDS that reside along with the micro imaging lens and sensor at the distal end of the shaft of the scope. Electrical wires run also through the length of the shaft to transfer electrical signals from the imaging sensor to the handle and ultimately to the image processing unit.

The sensor/camera preferably is rectangular, defining a rectangular image plane, not cropped to circular.

With the sensor rigidly attached at the distal tip of the instrument, shaft rotation will change the orientation of the image produced by the sensor. In the invention, the rotation of the shaft is continually sensed by the aforementioned rotation-sensing transducer. With the shaft rigidly attached to the forward portion of the handle, rotation of the shaft can be accurately sensed by the transducer. Thus, the rotational state of the shaft (and thus the sensor) is fed electronically in real time to the software/firmware performing image processing. This way the orientation of the image produced on a viewing monitor can be corrected in real time, by software or firmware applying an algorithm. The shaft is semi-rigid, and permits some limited bending, which is not possible with prior art devices that send an image through the length of the shaft from a distal tip optic back to a sensor near the handle.

The image processing electronics can reside inside the handle, but preferably are located remotely from the hand-operated instrument itself, receives the electrical signals from the electronic imaging sensor along with a continuing signal indicating orientation of the sensor. This rotation-sensing transducer can be an encoder, potentiometer, magnetometer, etc. With this information the processor can, in real time, restore the image from the sensor to normal, i.e. horizontal dimensions parallel to the horizon, and the viewer sees a video image stabilized in orientation even while the sensor is rotated.

The invention covers another important feature where the handle itself carries another set of electronics that is sensing spatial location of the full handle and that is separate from, and in addition to, the tip rotation-sensing electronics. This “extra” electronics could be sensing the instrument's rotation and tip, tilt, yaw, orientation to earth's magnetic axis and all other related spatial location measurements known in the state of the art. This “extra” electronics can be used in conjunction with the rotation measuring electronics of the tip in order to correct the motion of the full handle as it pertains to a surgeon or operator moving the handle in 3D space while at the same time he/she is rotating the chip-on-tip via the rotating mechanism of the handle. These measurements of the two separate systems (one manipulating the tip and reading the tip rotation with respect to the rest of the handle, the other reading the rotation and location of the rest of the handle in space) can be used differentially and in coordination in order to achieve the true rotated image in the monitor of the operating room that is free from any parallax, or extra shift in image rotation created by the handle motion.

The electronics of the scope handle orientation are located in a part of the handle that is not affected by the rotation of the tip of the scope. In some embodiments where the chip-on-tip is rotated by a mechanism at the front of the handle proximal to the patient, the second set of handle electronics is located at the fixed handle portion distal to the patient that is held firm in the palm of the operator's hand, while the fingers of the operator are manipulating the rotating mechanism for the chip-on-tip.

This invention also covers a third set of electronics and mechanical system that includes a form of a “brace” or “sleeve” that is fitted onto the patient at the location of surgery. This system could wrap around the knee or the shoulder so that it correlates the patient anatomy with the location in space (operating room x,y,z coordinates) of the specific body part. The electronic measurement of spatial location and rotation of the body feature of interest is transmitted into the handle electronics of the scope and/or onto the base system in the operating room. This information is used in a differential and coordinated manner with the other two measurements of rotation and location of (a) the chip-on-tip and (b) the handle, in order that a “true” image rotation correction of the anatomical features imaged by the scope can be achieved and then projected onto the monitor of the operating room or on a computer, or saved. Such a system for minimal guidance can be as described at 7Dsurgical.com, and can locate for the surgeon the proper point of entry on the patient, for the surgical procedure. On the monitor is shown the surgical tool's location and orientation, overlaid on an x-ray, MRI, CT or other scan shown on the monitor. This assumes proper surgical entry, after which the scope provides visualization of internal tissues. As an alternative to use of the patient's own scan the system can utilize a database of past images of similar anatomy and in turn display on a monitor both the current real-time image and a representation of all past images of similar anatomy. The database of anatomical images can be further optimized by entering a minimal set of patient data such as birthday, height, weight, sex.

The invention has several other important features. For rotation of the sensor-carrying shaft relative to the handle, a PCB slip ring can be employed, with brushes for continued electrical contact during rotation. In addition, the PCB slip ring can carry electronics for driving an LED light source, as well as other functions such as data from an NVram chip that can hold calibration information about the sensor and the disposable assembly as a whole.

The manner of assembly of the endoscope instrument is also an important aspect of the invention. The device preferably is held together without screws or threads, but only snap-together plastic components, firmly held together after assembly.

An endoscope is typically introduced into the body (especially in laparoscopic or arthroscopic procedures) through a cannula. Such cannula is typically outfitted with a liquid or gas access port (with typically a luer port fitting) so that liquid or gas can flow into the viewing area at the distal end of the cannula through the annular space between the OD of the scope shaft and the ID of the cannula. The cannula port should be free to rotate relative to the cannula body while still providing a sealed path for the gas or liquid infused to flow only through the cannula's annular space. A fluid-sealing slip ring is provided at the fluid port, so that rotation of the instrument can still be effected without hindrance of a moving/rotating fluid supply tube.

The invention offers the user of the endoscope the same feel as a “regular” or conventional side-viewing scope in which the fiber illumination port of the scope is used as a handle to rotate the front “lens” portion while the user holds the back portion of the handle that encompasses the image sensor. Orthopedic surgeons are specifically trained to use arthroscopes that have a 30° angle with respect to the forward looking direction such that the FOV of the scope is tilted by 30° to the side. Prior to the invention the prevalent use of this mode of operation has hampered the use of chip-on-tip devices in orthopedic practice. The invention specifically unlocks this novel use case.

Holding the full scope and rotating by a single hand operation is an important feature of the invention and is enabled by the instrument's structure. Conventional bulky devices are unable to be used in such a way.

True orientation correction of anatomical features imaged by the chip-on-tip scope is another important feature of the scope and is achieved by multiple spatial reading systems in the handle of the scope and the patient reference system.

Endoscopic procedures, and particularly arthroscopies and laparoscopies, are performed by inserting the scope into the body through a cannula, to which the scope locks on once fully inserted. There are two major reasons for that. One is to protect the scope during insertion (as the cannula is inserted first and thus provides a clear and safe path for the scope to enter the body), as well as to protect the scope during the operation while the cannula/scope construct is manipulated while already inserted into the body through tissue, since the cannula and not the scope will experience all the forces applied to the construct during manipulation. Another reason is to provide a way for infusing liquids or gas into the body through a side port on the cannula and then through the annular space between the inside diameter of the cannula and the outside diameter of the scope.

Another aspect of the invention is a disposable cannula that contains a luer port that can freely rotate in place with respect to the long axis of the cannula. Such rotating port allows delivery of liquids or gasses into and through the inner lumen of the cannula, while it can rotate freely around its place, and allows for better management of the liquid/gas line or syringe attached to it while the side-viewing scope is rotated for viewing new information. Multiple devices that achieve this rotation are either reusable, and constructed of relatively expensive materials and methods, or their rotation is done at a single cross section using a single O-ring or material that seals and allows for rotation. A preferred construction is a cannula that has two 0-rings to allow disposable grade materials, e.g. plastic to be used for the luer port rotating portion of the cannula, while balancing the forces of the rotation via friction between the O-rings on the main cannula body and the rotatable luer port. Additionally, such cannula provides the input infusion port proximal to the location of the imaging sensor. This is a unique feature compared to the typical arthroscopes or laparoscopes where such infusion port resides distal to the imaging sensor.

Embodiments of the invention further include several structural arrangements providing for disposability of the distal end of the scope, and several different structures and protocols to provide sterility of the surfaces of the scope that will be exposed during use of the scope.

A principal objective of the invention is to improve efficiency, accuracy and minimal invasiveness of endoscopic diagnosis and surgery with a very small-diameter, chip-on-tip rotatable scope providing for real-time correction of rotational orientation of the video signal from the imaging sensor.

### DESCRIPTION OF THE DRAWINGS

FIG. 1 is a perspective view of the instrument of the invention, connected to processing electronics/monitor.

FIG. 2 is an enlarged view of the endoscope alone, including a rotatable shaft with chip-on-tip.

FIG. 3 is a perspective view showing a disposable part of the instrument as separable from a reusable part, in a preferred embodiment.

FIG. 3A is a further enlarged view in cross section schematically showing details at the tip of the shaft for a forward looking version of the scope.

FIG. 3B is an enlarged end view, showing the distal end tip of the shaft.

FIG. 3C is a schematic end view of the shaft with an LED at the distal end for illumination, rather than optical fibers.

FIG. 3D is a close-up photograph in perspective showing the distal end of a needle or shaft with clip-on-tip.

FIG. 3E is a side view, partly shown transparent, of the shaft distal end, but showing the sensor and optic at an angle with respect to the longitudinal axis of the scope.

FIG. 3F is a perspective end view of the angle-view scope of FIG. 3E.

FIG. 4 is an exploded view showing component parts for assembly of the instrument.

FIG. 5 is a sectional view showing the assembled instrument and internal components.

FIG. 5A is an enlarged sectional view of the instrument body.

FIGS. 5B and 5C are exploded perspective views indicating assembly in one preferred form of the invention.

FIGS. 6 and 7 are detail views in perspective showing plastic parts, components of the instrument.

FIG. 8 is another perspective view, with some components shown transparent, showing a disposable distal end containing an LED and heat sink and indicating connection to a non-rotating handle.

FIGS. 8A, 8B and 8C are perspective drawings indicating a disposable distal end of the instrument and demonstrating assembly and removal.

FIGS. 9 and 9A show further details of the embodiment shown in FIG. 8, in a preferred embodiment.

FIG. 10 is an exploded view showing PCB slip ring components that can be included in the instrument.

FIG. 11 is a side schematic view showing gearing for an off-axis rotation sensor/transducer.

FIGS. 12A, 12B and 12C are side views of a gas/liquid cannula that can be assembled over the shaft of the instrument as in FIGS. 12D and 12E.

FIG. 12F is an enlarged side view showing the distal end of the instrument's shaft with the cannula installed, the instrument being side-viewing in this embodiment.

FIGS. 13A and 13B are exploded perspective and side views showing another embodiment of the invention with scope rotation functionality residing in the distal, disposable end.

FIGS. 14 to 25B are views showing other aspects of the scope construction shown in 13A and 13B, with some variations in different embodiments, and showing assembly of the two parts and with some of the figures indicating a rechargeable instrument and a charging dock.

FIG. 26 is a side sectional view showing a particular embodiment of the invention having outer shell components for sterile handling of the scope.

FIGS. 27 and 28 are elevation views showing the manner in which plastic component parts can be assembled, using the sterility shells such as shown in FIG. 26.

FIG. 28A includes three images illustrating an assembly similar to that of FIGS. 26 to 28 but with a variation.

FIGS. 28B, 28C illustrate a sterile cover or bag that can be unfurled over the instrument handle for sterile handling.

FIG. 29 is a logic/flow chart showing routine for detection of the angle of rotation during use of the instrument, and real-time correction of angular orientation by an image processor.

FIGS. 30 to 32 show a photographic demonstration of the use of the instrument of the invention with rotation of the distal end of the scope to show different regions on a sample positioned on a table surface, and resulting video images.

### DESCRIPTION OF PREFERRED EMBODIMENTS

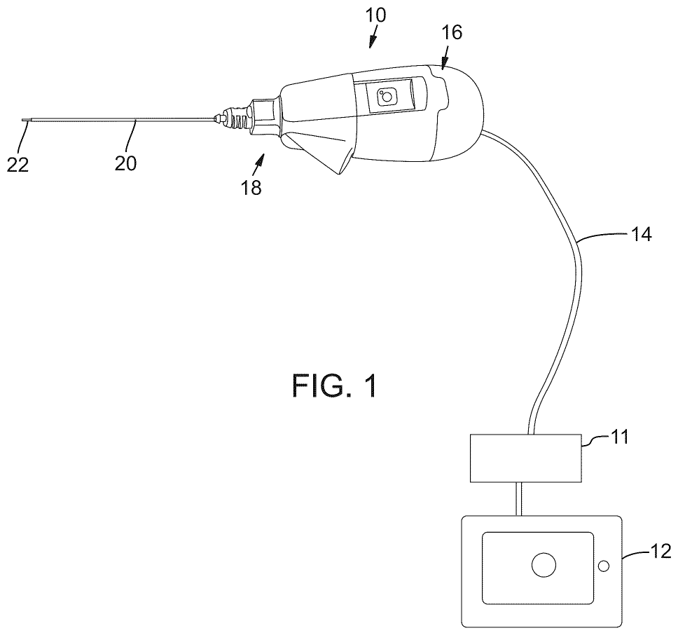

In the drawings, FIG. 1 shows a portable, handheld instrument 10, connected to processing electronics 11 and a monitor generally indicated at 12. Although a cable 14 is shown connecting the instrument to the electronics and monitor, this connection can be wireless, with a transmitter (not shown) in the instrument 10 and a receiver (not shown) at the processor 11, and with a battery in the instrument. The instrument 10 includes a handle with a base handle portion 16, a forward (distal) rotatable portion 18, a proximal part of which forms a part of the handle, and a scope shaft 20 which has at its distal tip 22 a sensor, optics for forming an image on the sensor, and the end tips of optical fibers carrying illumination generated by an LED in either the handle base portion 16 or the rotatable part 18. Alternatively one or more LEDs can be located at the tip 22.

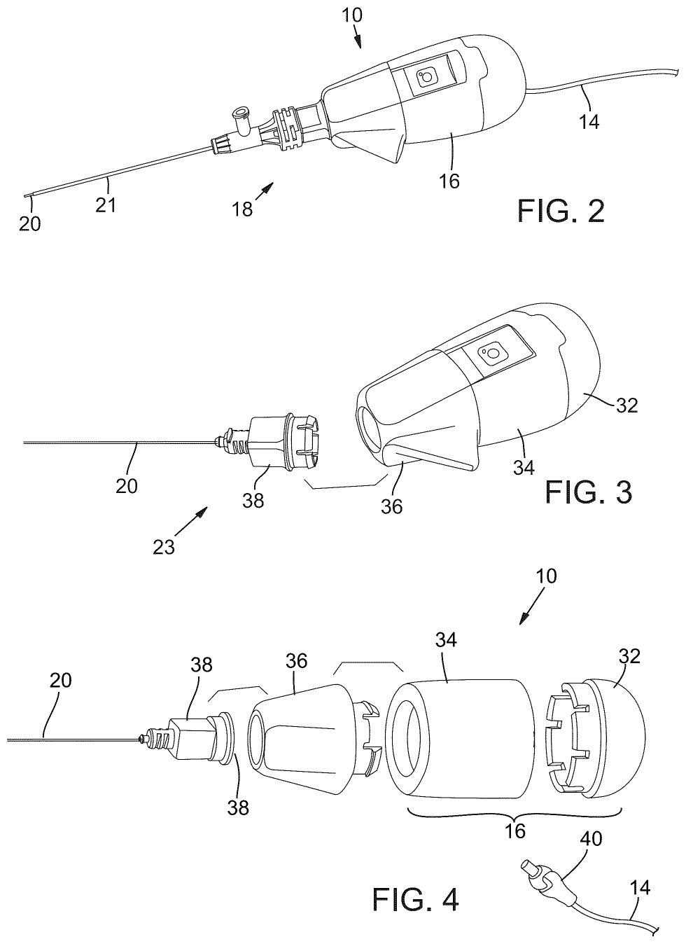

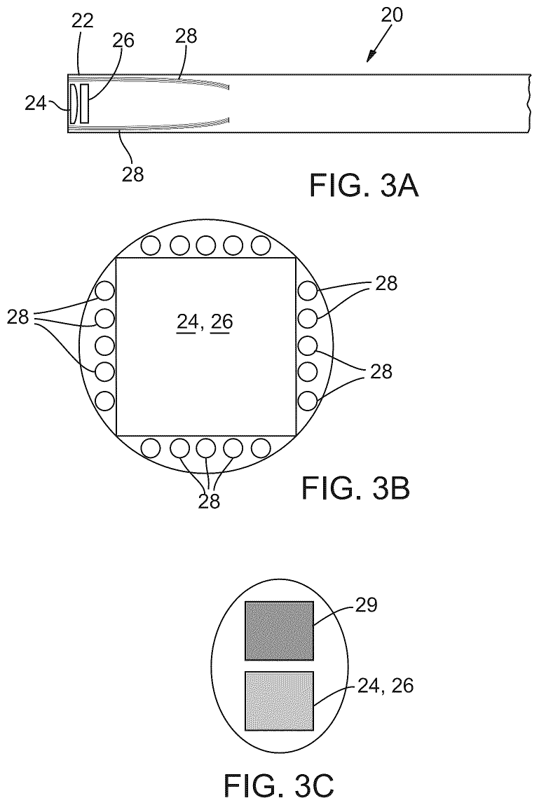

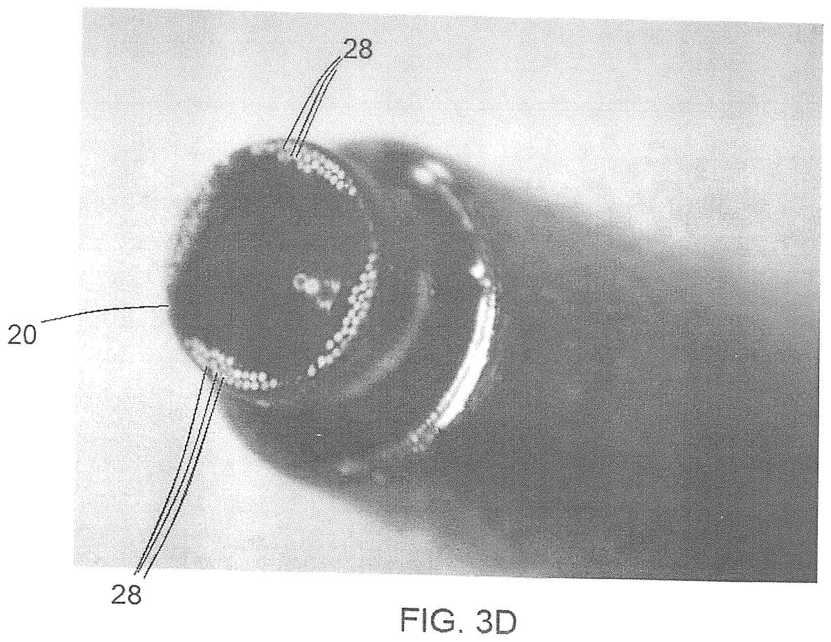

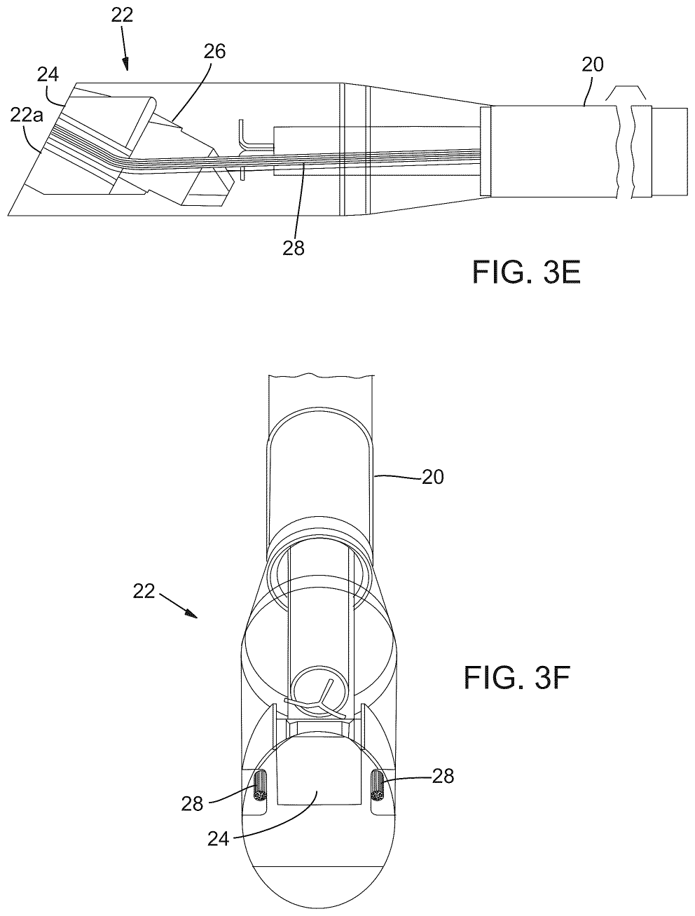

FIG. 2 shows in the endoscope 10 alone, with the shaft 20. The rotatable distal part 18 rotates with respect to the fixed base 16. In FIG. 2 a cannula 21 is shown assembled over the needle/shaft 20, with a luer port 21a. FIG. 3 schematically indicates an embodiment with a disposable distal section 23, separated from the reusable handle body including an end cap 32, a main body section 34 and a rotatable cone-shaped distal part 36. A needle retainer 38 holds the needle or shaft 20 and although disposable the retainer 38 is assembled rigidly (by snapping and un-snapping) to the rotatable cone piece 36 so that the components 20, 38 and 36 rotate together.

FIG. 3A shows the distal end of the shaft 20 in a zero-degree optical axis embodiment. The distal tip 22 is shown in cross section. The distal tip 22, preferably about 1.4 mm O.D., carries an imaging optic(s) 24 which forms an image on a sensor or camera 26, both fixedly mounted in the tip (“chip-on-tip”). Optical fibers 28, also shown schematically in the end view of FIG. 3B, provide illumination originating at an LED located proximally in the handle. FIG. 3C illustrates an LED 29 at the distal tip of the scope shaft, as an alternative to optical fibers. The distal tip may be an oval or ellipse with the LED located there, although it could also be circular.

FIG. 3D is a photo image of the distal tip, showing a large number of optical fiber distal ends 28 located along sides of the square sensor/optics 24, 26. In this view a cannula is shown positioned over the shaft 20.

FIGS. 3A-3D show a forward-looking, zero-degree scope, while FIGS. 3E and 3F show a side-viewing scope. In FIGS. 3E and 3F, rendered transparent for purpose of clarity, the distal end 22 is shown somewhat enlarged from the remainder of the shaft or needle 20. The distal lens-sensor-illumination assembly is preferably beveled as shown, for the side-viewing scope such as a 30° side-viewing scope which can be constructed as illustrated. As in the 0° embodiment, the distal tip face 22a is flat, the distal-most element being the optic, typically a lens. Fiber optics, i.e. a bundle of fibers, are shown at 28, not being at center but on sides, as shown in FIG. 3F (but preferably all bundled together proximally). The imaging optic is seen at 24, with the camera/sensor 26 immediately behind, i.e. in the proximal direction. Both the optic 24 and the camera/imaging sensor 26 are oriented with their optical axis at an acute angle from the longitudinal axis of the instrument, e.g. 30° as shown here. The illumination from the fibers is always aimed in the same direction as the imaging sensor, which is an important feature.

Although not shown here, another feature of the invention is that the fiber optic 28 ends can be bevel-cut to establish at least a part of the angulation at the tip of the scope, to bend the light rays as desired. In one embodiment the fibers are bent (curved) partially toward the desired angle, and the remaining redirection of light is via bevel cuts at the tips of the fibers.

FIG. 4 shows, in exploded view, the main components of the portable endoscope 10, with indication of their assembly according to one form of the invention. The housing or base handle part is indicated at 16, including the rear cap 32 of the base portion and the main body 34 of the base portion. In this embodiment the forward cone piece 36, a part of the handle, is connected to the forward or distal end of the base 16 so as to be rotatable relative to the base, by hand rotation. Fixed to the cone 36 is the rotatable shaft retaining component 38, rotating along with the shaft 20. Note that the shaft retaining component 38 could itself be rotatably connected to the body 34, as in another embodiment described below, with the cone piece 36 simply secured over the component 38 for rotation together. In another embodiment (as in FIG. 3) the components 20 and 38 can be disposable, removable from the cone piece 36. The connections of the components 38 and 36 is made by snapping them together, with a proximal end 38a of the needle retainer 38 snap fitting into the distal end of the cone piece 36 in this particular embodiment, the connection being secure and non-rotatable. The instrument's cord and an end connector are shown at 14 and 40.

All components 32, 34, 36 and 38 are configured to snap firmly and reliably together, without screws or threaded features, and with rotation afforded between the parts 36 and 34. This construction is preferred for simplicity and reliability. The connections are secure and tight, preferably designed to lock with some kind of standard ingress protection level, such as IPX4 including the rotatable interface between parts 36 and 34.

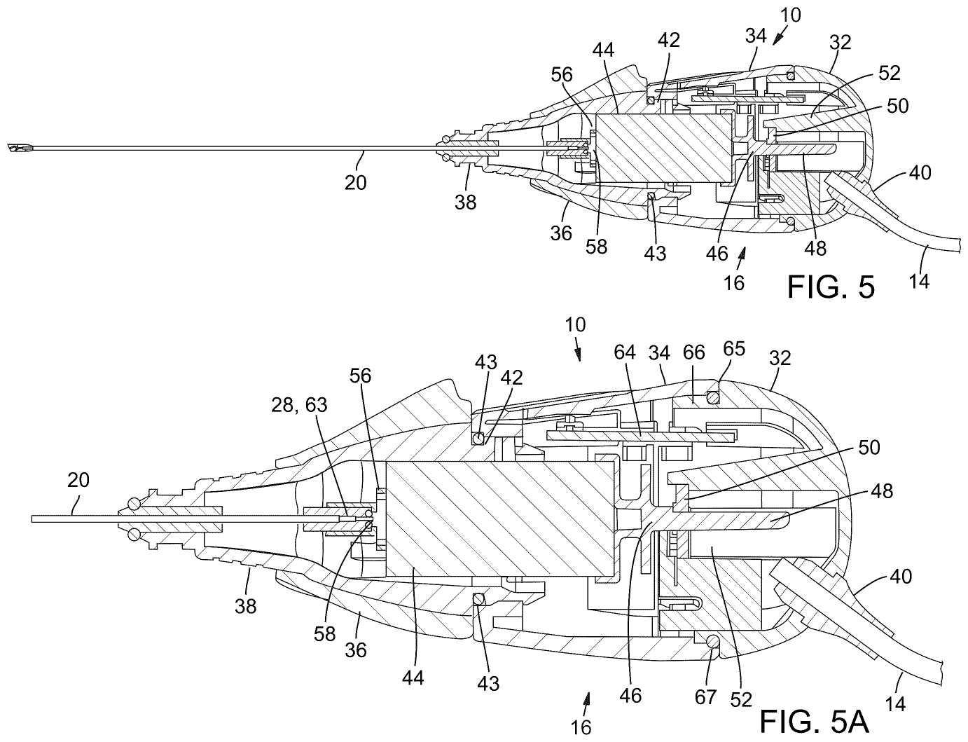

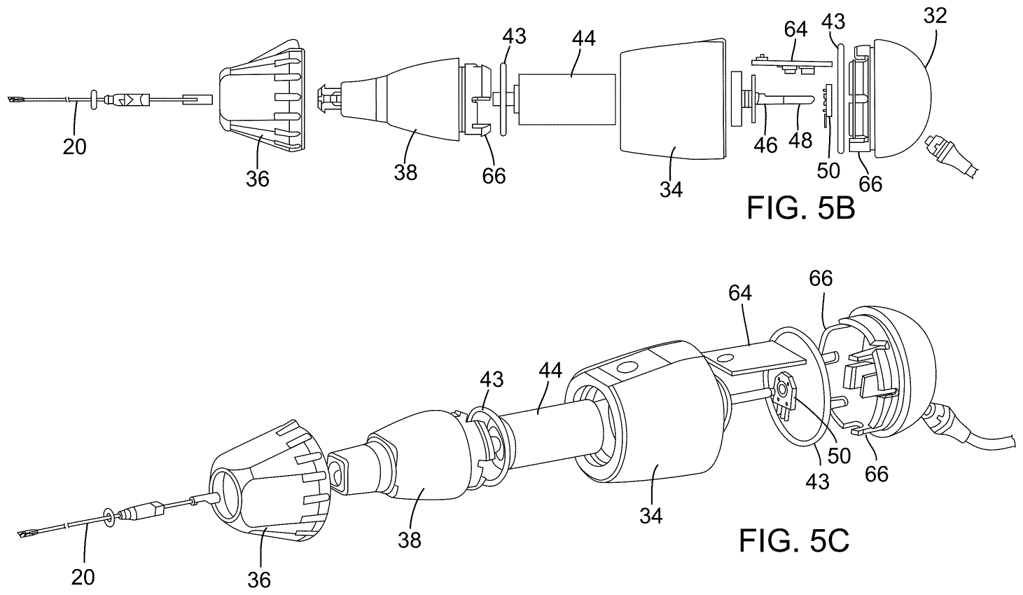

FIGS. 5 and 5A show the instrument 10 as assembled, with some detail of internal components in a preferred embodiment. In FIGS. 5 to 5C the instrument is either fully disposable or fully reusable (as by autoclaving between uses).

As indicated, the needle shaft 20 is fixed to and rotates with the shaft retaining component 38 which is secured to the distal end of the instrument body, i.e. to the forward cone 36. The cone 36 rotates with the shaft and retaining component 38, relative to the base handle portion 16. Extending into the base portion 16 is a collar 42, integral with the forward cone 36. A sealed rotation connection is made between the components 36 and 16 via the extending collar 42, shown with an O-ring seal 43 sealing this rotatable connection. In the embodiment shown the collar 42 has connected to it a cylindrical heat sink 44, all of these components 20, 38, 36, 42 and 44 rotating together relative to the base part 16 of the instrument.

In this form of the invention a potentiometer adapter shaft 46 extends proximally from the heat sink 44 and is rotatable therewith (the illustrated additional proximal length 48 on the shaft 46 is for assembly purposes). The adapter 46 extends into and engages firmly with an inner rotatable component (not specifically shown) of a potentiometer 50. When the forward conical piece 36 and needle shaft 20 are rotated, which is fixed in position within the base 16, shown as secured to structure 52 integral with the housing component 34. When the forward cone piece 36 and needle shaft 20 are rotated, this rotates the inner component of the fixed potentiometer 50 to generate a signal representing degree of rotation. Wire leads (not shown) extend from the potentiometer chip or PCB 50, for connection to further electronics and power, and ultimately the signal is sent to the image processor and to the monitor, either via the cord 14 or wirelessly.

The heat sink 44 is secured in contact with an LED driver PCB 56 within the forward cone-shaped piece 36, with an illumination LED 58 being mounted on that PCB as indicated schematically in the drawing. LEDs generate considerable heat and require some form of heat sink. The heat sink could alternatively be located in the cone piece 36, possibly with a heat-conducting path to the exterior of the cone piece.

Against the LED, preferably in direct contact, are proximal ends of the optical fibers 28. There may be many such fibers as noted above. Electrical wires 63 are also included in the bundle entering the needle shaft 20 along with the optical fibers 28.

FIGS. 5 and 5A further show a main PCB 64 retained securely within the housing, specifically fixed within the body section 34 as shown. The wire leads 63 extending proximally from the needle shaft 20 ultimately are connected to the PCB 64, via the rotation-accommodating connection.

In lieu of the potentiometer a magnetometer could be used, whereby a relative rotational movement of a magnet is sensed for orientation, but in the case of a magnetometer, the proximal end of the potentiometer adapter shaft 46 has a magnet in near proximity to the magnetometer.

FIGS. 5 and 5A also indicate the snapped-together assembly of the components, which were shown in exploded view in FIG. 4 and also in FIGS. 5B and 5C. A connection between the rear cap 32 and the body 34 is made at 65, with latching tabs 66 or an annular latch engaging at the annular proximal end of the body section 34. An O-ring 43 provides a seal. On assembly the latching structures 66 of the end cap snap into corresponding grooves or recesses in the body 34 to make a permanent connection.

FIGS. 5B and 5C further illustrate assembly, with internal components in this preferred embodiment. The connection of the shaft-retaining proximal piece 38 to the cone shaped piece 36 is made by advancing the forward shaft retaining piece 38 through the cone piece 36, with an O-ring 43 in position as shown in FIGS. 5 and 5A. As can be seen in FIGS. 5 through 5C, in this embodiment the cone shaped piece 36 is actually secured to the needle retention piece 38, rather than being secured to the body section 34. Thus, the cone shaped piece 36 rides along with the needle retention piece 38, the rotation being afforded between the components 38 and 34, with the O-ring 43 sealing that rotatable connection. In FIGS. 5B and 5C retention tabs 66 are shown in an annular array at the proximal end of the shaft retention piece 38, for locking engagement with the distal end of the body section 34, for a secure but rotatable connection therewith. Similar retention tabs are shown on the end cap 32, with another O-ring 43 at that location.

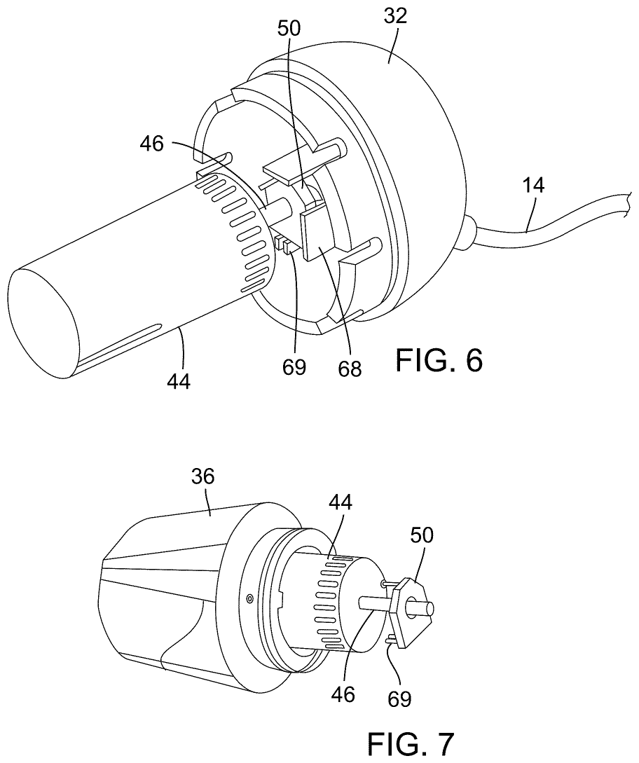

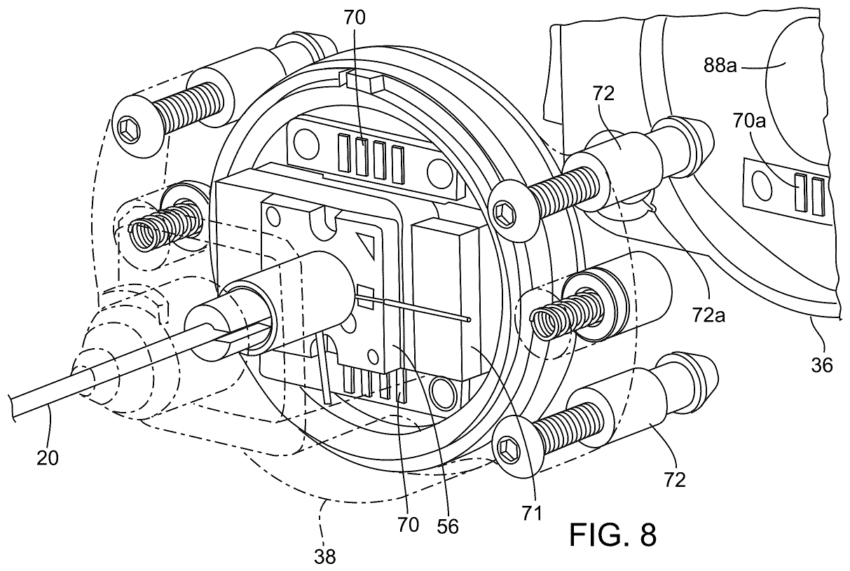

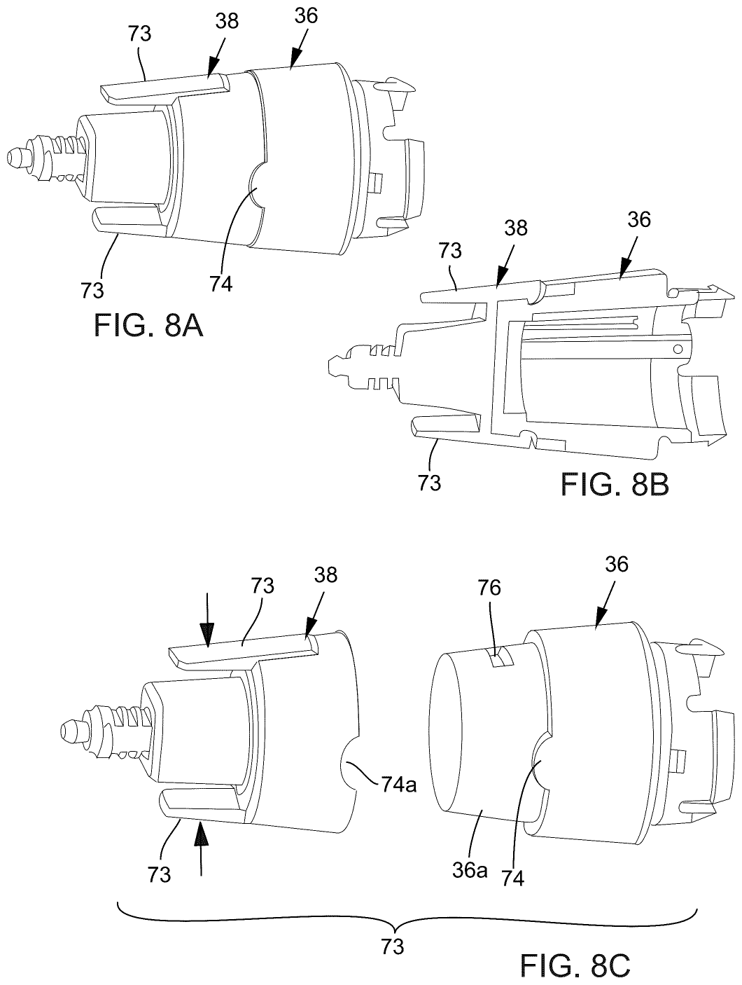

FIGS. 6 and 7 show details for one preferred embodiment of the invention, particularly regarding the transducer that determines rotation of the imaging sensor and optics relative to the main body of the handle 16. Again the rotation-sensing transducer is shown as a potentiometer, at 50 in FIG. 7, attached to the fixed portion 32 of the handle (in FIG. 6). The same heat sink 44 is shown attached to the rotating cone piece 36, for dissipating heat from the LED, as in FIGS. 5 and 5A. It is noted that such cylindrical heat sink 44 may not be needed in the event the LED is located in the shaft retention component 38 (as in FIG. 8 below), but the potentiometer adapter shaft 46 nonetheless will be included, rotating along with the forward cone-shaped piece 36, the shaft retention piece 38 and the shaft 20. This transducer or adapter shaft 46 is shown extending through the potentiometer 50. This way the resistance of the potentiometer 50 that is securely held in the cap 32 (FIG. 6) can vary as piece 36 is rotated and thus the potentiometer's resistance can be translated to a specific rotation through a simple calibration at the factory, to indicate the rotational position of the camera/imaging sensor and optics. The signals indicating rotational position are derived from changes in a voltage, due to changes in resistance of the potentiometer, due to different rotational positions of the shaft relative to the potentiometer 50.

FIGS. 6 and 7 indicate a preferred form of securement for the potentiometer 50 within the housing, i.e. within the rear (proximal) cap 32. In FIG. 7 a form of seat 68 is shown as a raised boss or flange integral with the rear cap at its interior. This seat has a shape that mates with that of the potentiometer 50. FIG. 6 indicates the potentiometer 50 in place in the seat 68 of the rear cap 32 and middle section 34. FIGS. 6 and 7 also show electrical contacts 69 of the potentiometer, which will make contact with the PCB board (not shown) within the rear cap 32.

In another preferred form of the invention, the needle 20, including the shaft with distal-end sensor and optics, and illumination optical fibers 28, and including the shaft retention component 38, are disposable, while all structure seen to the right (i.e. rotating cone piece 36 and handle 16) is reusable. See FIG. 3 as an example. In one embodiment the LED can be included in the reusable portion, thus requiring a coupling of light from the LED to the optical fibers 28 that extend to the tip of the shaft. This requires a precise coupling, with the fibers in contact with an internal component of the cone 36 that receives light from the LED, and it is preferable that the proximal ends of the optical fibers 28 make actual contact with an LED light-transmitting component within the cone 36. Preferably some form of spring is included to allow the fibers to be in such rotatable contact. In one aspect, the optic fibers 28 can themselves act as a spring, by including a slight buckle in the fibers such that the buckle curvature can be increased as the fibers press against the component in the reusable section, buckling back slightly.

However, in another preferred construction the LED is located in the disposable section, i.e. within the shaft containing component 38. This is shown in FIG. 8. In FIG. 8 the LED 58, not shown, is mounted on a PCB 56 within the component 38. Optical fiber 28 ends (not shown) receive illumination from the LED, and wire leads (also not shown) from the shaft 20 extend to electrical/data terminal connectors 70 to interface with mating connectors 70a on the cone piece 36, which is not disposable and part of the handle. The wires carry power to the LED and data from the chip-on-tip sensor. The PCB 56 in this case is in contact with a smaller heat sink 71, which is contained within the shaft retaining component 38 as indicated. The arrangement can be such that the entire body of the shaft retaining component serves to dissipate LED heat, and in this regard the shaft retaining component 38 could be formed of metal, or could include metal (for clarity it is shown with transparent parts in FIG. 8).

FIG. 8 also indicates an example of a mechanism that can be employed for attaching the shaft retainer 38 to the proximal rotatable core piece 36, for releasable attachment. Barbed studs 72, permanently secured to the shaft retainer 38 (as by bolts as illustrated) are snapped into receiving holes (one seen at 72a) in the distal end of the cone piece 36, gripped with latches in the cone piece so as to hold the components together with the electrical connectors in reliable contact. A springable release (not shown) can be provided in the cone piece 36, to release the barbed studs after use of the needle.

FIGS. 8A, 8B and 8C show another form of releasable latch as an alternative to that of FIG. 8, for securing the disposable needle retaining piece 38 to the cone piece 36. In this embodiment the needle retaining piece 38 has springable plastic levers 73 with barbed proximal ends for engaging into recesses of the cone piece, as best seen in FIGS. 8B and 8C. The exploded view of FIG. 8C illustrates the assembly/disassembly of these two components. When the attachment is made, the needle retaining piece 38 (with the needle shaft, not shown) is pushed onto the cone piece 36 thus sliding over a collar 36a of the cone piece. Correct orientation is achieved by a bump or boss 74 on the cone piece, providing for only one orientation as the two are assembled, nesting into a detent 74a. As the needle retaining piece 38 is slid onto the cone piece 36, the springable levers snap into the recesses 76 in the collar 36a. This deflects the lever 73 as the piece 38 is slid over the collar 36a, then the levers snap firmly into place at the point of full assembly. Again, this assembly engages electrical connectors together between the components 38 and 36. For removal of the needle and needle retaining piece 38 after use, the distal ends of the levers 73 are squeezed toward one another, as indicated in FIG. 8C, to enable the piece 38 to be pulled free of the cone piece 36.

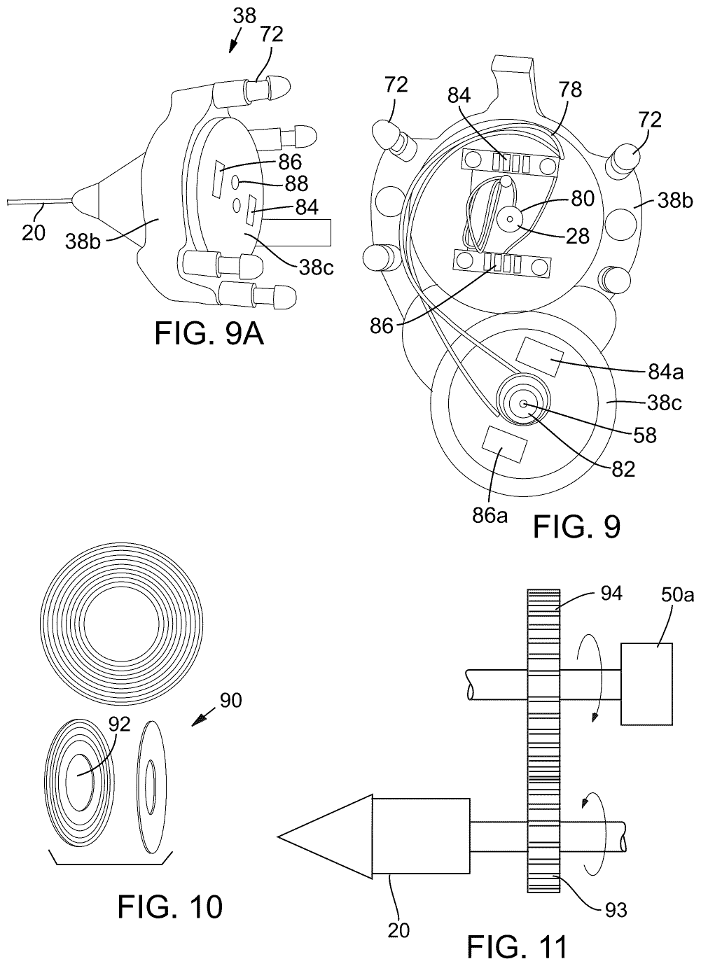

FIGS. 9 and 9A show one preferred example of a construction within the shaft-retaining piece 38, for the case in which the LED is located in the disposable part of the instrument, as in FIG. 8. FIG. 9A shows a distal piece 38b of the assembly, the needle or shaft 20 being secured to the distal end. A second, proximal piece 38c is shown secured to the piece 38b in FIG. 9A, forming the shaft retention piece 38 essentially as shown in FIG. 8. FIG. 9 shows the same two components in an exploded view, disassembled. The components 38b and 38c are permanently secured together as shown in FIG. 9A, as the distal piece 38.

In FIG. 9 at the center of the component 38, i.e. lying on the longitudinal axis and coincident with the shaft or needle 20, is a fiber optic end 28 that makes an interface with the LED 58 (seen in FIG. 9A) which is mounted in the center of the other component 38c. Wiring 78 connects to the LED, lying within the space defined between the components when assembled. Inner wires shown near the fiber optical 28 come from the imaging camera. As shown, a cylindrical stud 80 around the fiber optic end can be provided to fit within a socket 82 adjacent to the LED 58, for correct registry of the two components and the LED and fiber optic or fiber optic bundle end.

FIG. 9 also shows metallic contacts in a contact pad 84 at one side of the component 38, and additional contacts 86 at an opposite side. These are positioned on opposed sides of the longitudinal axis of the instrument. The contacts 84 are provided for camera control and readout data while making contact with mating contacts 84a on the other component 38c. The connectors 86 are for LED illumination control, and when the components 38b and 38c are secured together, these act with contacts 86a on the component 38c. An example of elastomeric connector is from the Z-axis Connector Company at web-link: axisconnector.com/products/z-wrap/.

These elastomeric core and wire wrapped connectors can be adapted to make contact with any number of circuit board pads that for example can address 10 or 20 or 100 of each set of wires.

In the assembly of the two components shown in FIG. 9A, the proximal side of this assembly continues the connections for LED illumination control (at 86) and for camera control and data readout (at 84). A heat sink extends from contact with the LED 58, i.e. contact with the PC board on which the LED is positioned, and is exposed at 88, centrally of the assembly. When the assembled needle retention piece 38 is secured to the cone shaped piece 36, in a connection between disposable and reusable parts of the instrument, the heat sink at 86 makes solid contact with a heat sink component 88a of the reusable part of the instrument, such as shown in FIG. 8.

FIG. 10 shows, in an exploded perspective view, a form of slip ring 90 that can be used in the device of the invention. As discussed above, a rotational connection must be made between the rotatable components 20, 38, 36 and the non-rotatable handle portion 16. The electrically connecting slip ring set 90 illustrated in FIG. 10 is manufactured by MOFLON company of Shensen, China. The rings have contacts that ensure a consistent connection of multiple electrical pads, which include power and data connections for the camera/imaging sensor and may also include power for the LED, assuming the LED is located in the disposable section of the instrument. The particular slip ring set 90 shown has a central hole 92, which makes it well adapted for this purpose, since the hole can receive components such as a rotational component for use in the transducer (e.g. the collar 42 shown in FIG. 5, not to scale), and any other on-axis components within the rotatable cone piece 36 such as portion of the LED heat sink 44 that could be shaped to fit in the hole of the slip ring. This preferably is a slip ring that can accommodate high bandwith electrical transmission from the optical CMOS chip to the on-board PCB. The slip ring allows the rotation of the needle to be more than 360° with respect to the back of the handle without twisting the wires and so allows for large viewing FOV's. Coupled with a multiturn potentiometer or a magnetic encoder that consists of a Janus magnet and separated as an example by air gap from the encoder circuit board allows for accurate measurement of angles greater than 360° and correcting the patient anatomy in the video display of the monitor or tablet hardware.

In another embodiment of the invention the tip can be rotated fast (e.g. with a motor, not shown). Fast rotation of the tip, with angled view allows for a continuous larger FOV not otherwise achievable. This has advantages in robotic vision to allow for large area anatomies without any loss of high quality imaging. For example it can substitute fish-eye lenses for cameras where equality of the realized image is compromised for large FOV's. Note also that for a zero-degree scope with square format the rapid rotation will present a larger, circular FOV with diameter equal to the diagonal of the square.

In another embodiment of this invention, an off-axis method of measuring rotation of the chip-on-tip needle shaft is provided. See FIG. 11. In this arrangement, a displacement measurement device located off-axis, such as an encoder, magnetometer or potentiometer, or optical device, is calibrated to translate displacement into relative rotation between the two surfaces where the displacement sensor is located. The rotation of the scope needle is transferred off-axis via gears 93, 94 to be measured by a potentiometer 50a or other device. One method is an optical reflection method via infrared sensors that are well-known in the art, e.g.

KEYENCE Corporation of America, Itasca, Illinois, sensors and as a specific example, sensor IL-030. In another example a mechanical method of displacement is calibrated to a rotation angle readout method, for example using a displacement sensor that employs an LVDT (linear variable differential transformer) or a digital contact device such as KEYENCE Corporation GT2 series sensors. In another example a time of flight (TOF) sensor can be sued to measure displacement. Resolution of the rotation angle is key to a good measurement and consistent viewing of the rotating video during operation. Also, small size of the handle can constrain the size of the displacement sensor used and that is a consideration in choosing from the list of the above examples of displacement sensors.

Another feature of the invention is provision for sealing the two relatively rotational parts of the scope handle while at the same time allowing for that relative rotation. Since this could also be a disposable scope that can have lower cost components, a less elaborate mechanism is preferential. In the embodiment described above, at least one O-ring 43 is placed between the two components and seals the spacing while at the same time is able to allow via reduced friction the rotation around the longitudinal axis of the instrument. The two components stay in place horizontally with respect to each other via a mechanism that can also be tuned to optimize the friction on the O-ring. In one embodiment, tension is applied via holding the back component onto the main axis via a screw loaded on a spring structure. The screw is threaded onto the potentiometer screw that can rotate with respect to the body of the potentiometer. The body of the potentiometer is placed firm on the axis and firm with respect to the front half of the handle. In other embodiments, the O-ring is replaced by a simple plastic or sheet band that joins the two halves to allow for both sealing and the necessary movement between the two halves. For small rotating angles, this can be accomplished by a small flexible material such as sterile cylindrically shaped tube made from thin plastic material such as a bag, positioned between the two halves. Glue, epoxy or hot-gluing or other means of joining the material to each of the halves are examples of assembling the system together. The loose material allows for twisting and rotating the one half with respect to the other up to a point where the material prevents further rotation beyond a specified angle. At the dimension of a few centimeters diameter of each of the halves, a wadded cylindrical bag of a length of 10 or 20 or 30 centimeters while stretched, can accommodate a rotation of plus and minus 180° with ease. In another embodiment the two halves are overlapping, with their overlap enabling sliding and rotating with respect to each other or with the help of an O-ring between them.

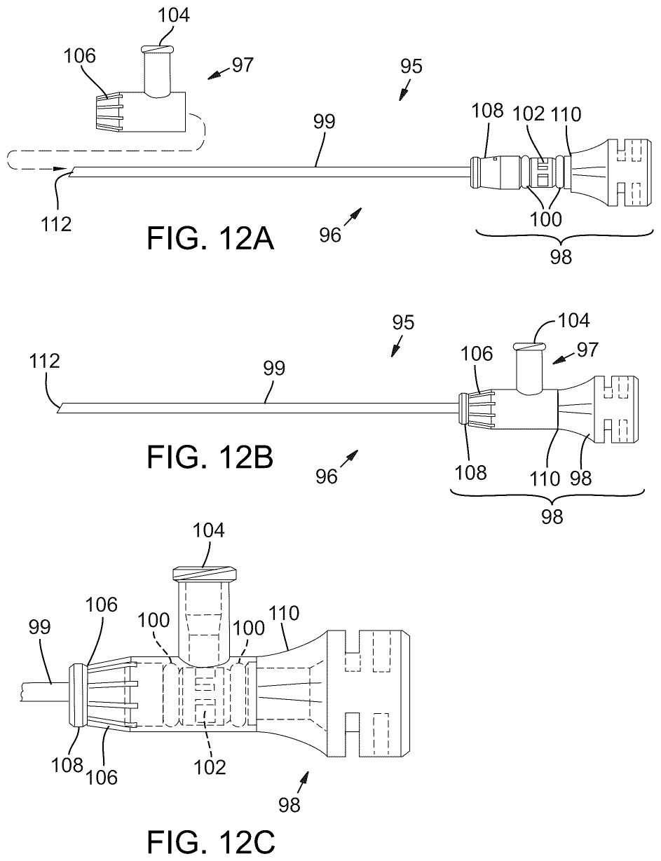

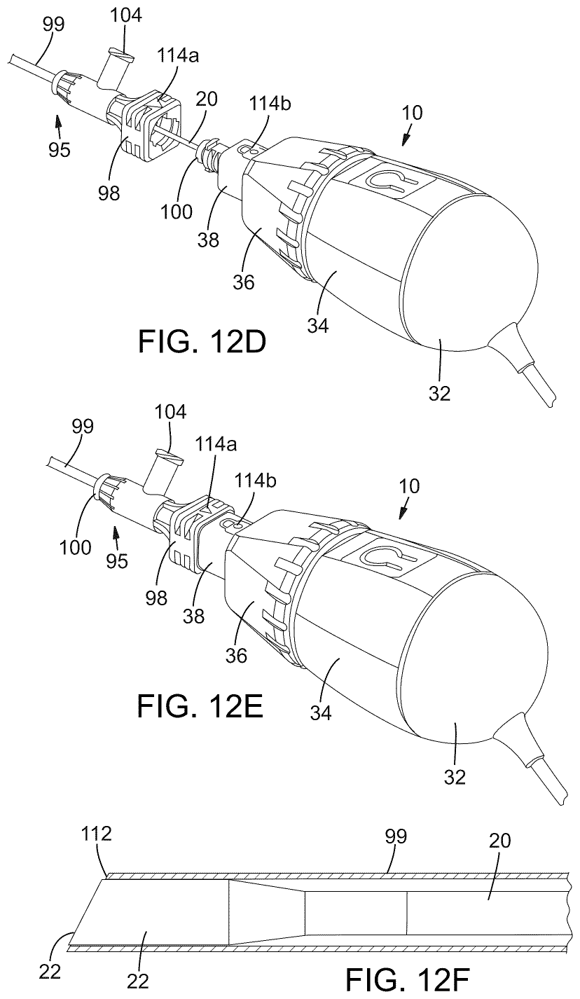

Another aspect of the invention is indicated in FIGS. 12A-12F, wherein a disposable cannula 95 is included.

The cannula 95 consists of two components: (1) the main cannula body 96, and (2) the rotatable luer port 97. The main body 96 is preferably constructed by overmolding a plastic proximal insertion port base 98 onto a metallic cannula shaft 99. Alternatively, the whole main body 96 (both 99 and 98) can be made from plastic pieces that are fused together. The rotatable luer port 97 is made of a plastic material and is designed in a way so that it can easily slide longitudinally over the cannula shaft 99 and be pushed over two O-rings 100 into its final resting place, as in FIGS. 12B and 12C. The two O-rings 100 are strategically mounted on the insertion port base 98 (distally and proximally from a region of the insertion port that contains a communicating hole 102, but preferably a multitude of holes communicating with the inside of the cannula. The rotatable luer port 97 can then lock in position longitudinally. In this assembled resting place, the rotatable luer port 97 can freely rotate around the main body 96 and have an interference fit with the two O-rings 100 and provide a seal for the fluids (liquid or gas flows) that are infused through the luer input port 104, while remaining longitudinally locked in place as in FIGS. 12B and 12C. The luer input port 104 can have an industry standard luer connection or any other thread or locking mechanism that will ensure the proper flow of liquids or gasses through the luer port. Although in FIGS. 12A and 12B the rotatable luer port 97 is shown with only one luer input port 104, it can also be made to have two such luer input ports.

Provision for the rotating luer port 97 lock into place longitudinally while being free to rotate in its assembled position can be designed as follows in this preferred embodiment: The rotating luer port 97 is further constructed with unidirectional features such as flexure ends 106 shown in FIGS. 12A-12C, so that when it is longitudinally pushed into position over the distal end of the main body 96 and locked in place over the insertion port base 98, it cannot be moved or pushed out in the opposite direction. It can only rotate in place. The flexure ends will open up when the luer port 97 is pushed into place (distal to proximal) and then collapse down when the luer port 97 has reached its final position and interfere with a carefully designed annular wall edge 108 distally on the insertion base 98 and a proximal wall 110. Such distal and proximal wall structures will prevent the rotating luer port 97 from moving longitudinally. See FIG. 12C.

The completely assembled cannula 95 is inserted over the shaft 20 of the side-viewing scope 10, as in FIG. 12D, and locks in place as shown in FIG. 12E. The insertion port base 98 has means for mating with the nose piece 38 of the scope and will lock securely with it in a specific orientation, so that the angled plane of the scope's distal tip 22 aligns with the angle cut plane of the distal tip 112 of the distal cannula shaft portion 99 in order that the cannula body not obscure the view of the sensor when the two are connected. See FIG. 12F. Such alignment of the two planes is imperative for a side viewing scope. Indication symbols 114a and 114b on both the proximal end of the cannula insertion port base 98 and the side-viewing scope's nose piece 38 can help the user orient the two components during the locking of the cannula 95 onto the scope 10, as in FIGS. 12D and 12E. An additional O-ring 100, FIG. 12D, can reside on the distal nose piece 38 to seal the interface between the nose piece 38 and the insertion port base 98 (when the scope and cannula are locked together) and prevent back flow of gasses or liquids through that interface when an infusion of liquids or gasses is taking place through the luer input port 97 (and thus force the flow to the distal tip 112 of the cannula shaft portion 99. An O-ring or similar sealing mechanism can also reside inside the proximal end of the insertion port base 98.

In all the embodiments of the side-viewing scope described so far (in FIGS. 1-9), whether the distal nose piece 38 of the scope (that is rigidly connected to the scope shaft 20 that contains the imaging sensor) is permanently attached to the scope handle or not, they all incorporate the rotation functionality of the side-viewing scope shaft 20 (and thus the sensor) in the handle (item 18 in FIG. 1 or item 36 in FIG. 4). In other words, in all embodiments described so far, it is item 36 or item 18 (the distal most part of the handle) that allows the user to rotate the sensor by rotating such distal handle pieces with respect to the rest of the handle (pieces 34 and 32 in FIG. 4).

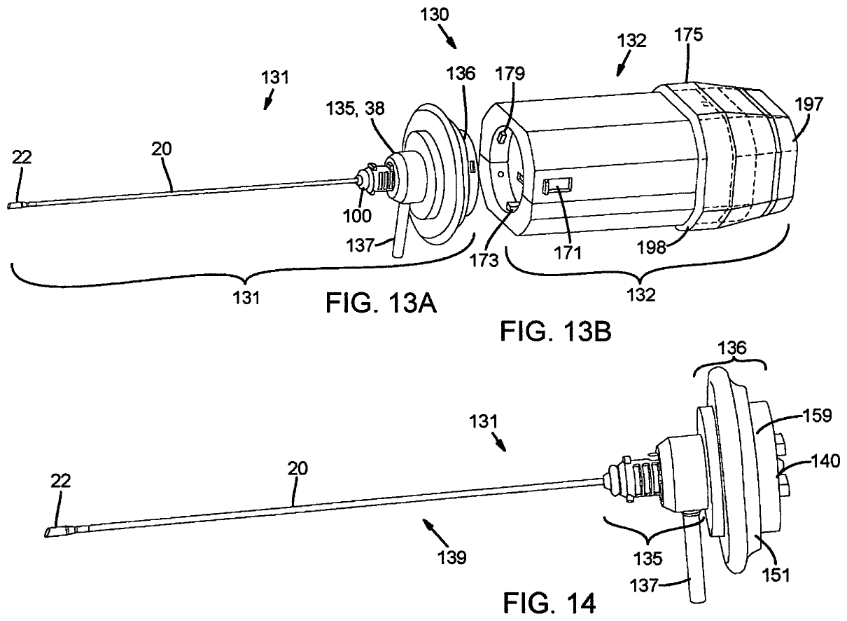

In another embodiment 130 of this invention (FIG. 13 through 25), the rotation functionality can reside on the detachable and thus disposable portion of the scope, and not in the handle. See FIG. 13. Thus, in this embodiment the detachable scope 131 (that also contains the rotation element 135) is disposable, while the handle 132 it connects to is reusable. Furthermore, in this embodiment it is preferable to have the handle 132 be communicating wirelessly with the video processing hardware 11 and monitor 12. However, in specific embodiment video data from the imaging sensor can be sent wirelessly to the processor 11 from the rotation element 135 via a transmitter located in that element or in the attached fixed element 136.

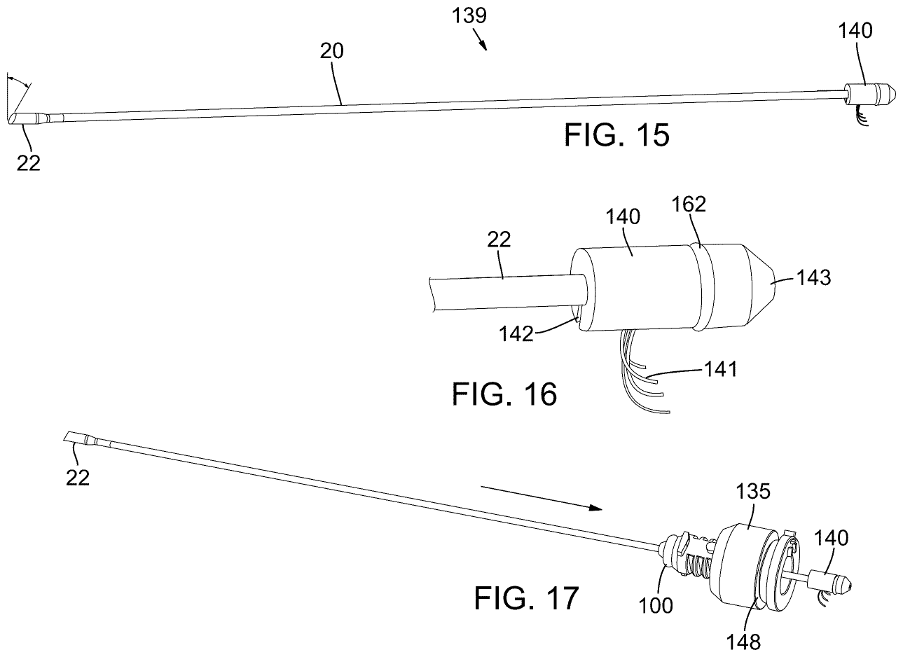

The detachable scope 131 is further shown in FIG. 14. It consists of three distinct components: (a) A small diameter assembly 139 that consists of the metallic shaft 20 containing the imaging sensor and lens at its distal tip 22 and a fiber termination tube 140 on its proximal end (as described previously, also see FIG. 15); (b) A distal rotating scope cavity element 135, that is in essence a modified version of the nose piece 38 of the previous embodiments and that also can mate with the cannula 95 previously described in FIG. 12—this is the part that rotates in this embodiment—the distal rotating scope cavity 135 is preferably equipped with a preferably removable lever 137 for ease of handling while rotating; and (c) The proximal fixed element 136, which is the component of the detachable scope 131 that mates with the reusable handle 132 and is fixed and non-rotatable once connected to the reusable handle 132.

As shown in FIG. 15, the metallic shaft 20 connects the distal tip 22 to the proximal fiber termination tube 140. At the distal tip 22 the imaging sensor and imaging lens reside with an angle cut of the end face equal to the side-viewing design angle of the scope. The distal tip assembly 22 is described in greater detail above, FIGS. 3E and 3F.

The fiber termination tube 140 is affixed to the proximal end of the metallic shaft 20 and as shown in FIG. 16 is designed with a longitudinal cut 142 so that electrical wires 141 can come out of the proximal end of the metallic shaft 20 and out of the fiber termination tube assembly for further connection to electronics, while the light-conducting fibers can be terminated straight through at the proximal end 143 of the fiber termination tube 140. The wires and light fibers run through the ID of the metallic shaft 20 from the distal end. The two end faces of the finished subassembly in FIG. 15 can now be prepared in any way that can provide good optical quality imaging and light transmission. Both end faces of 139 for example can be polished.

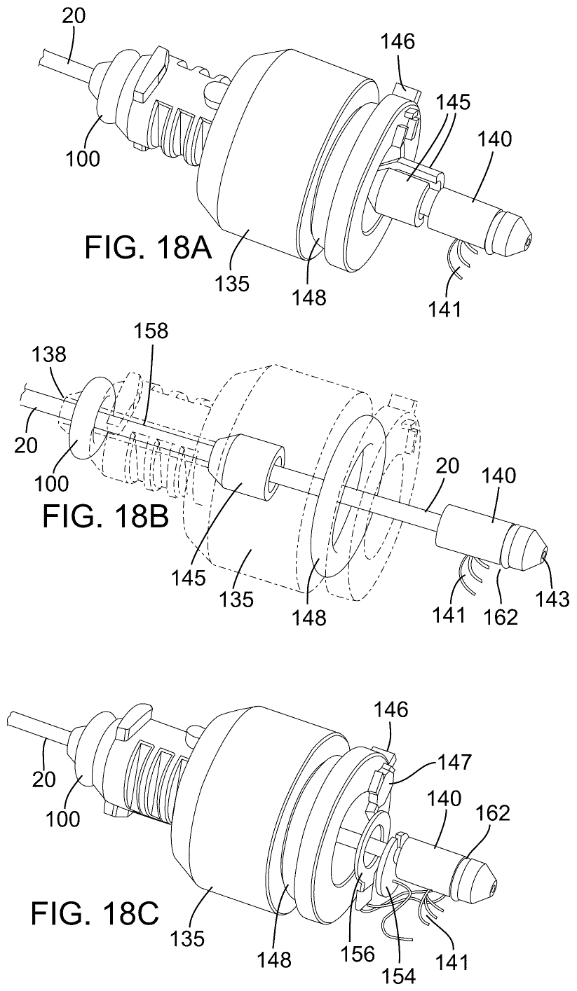

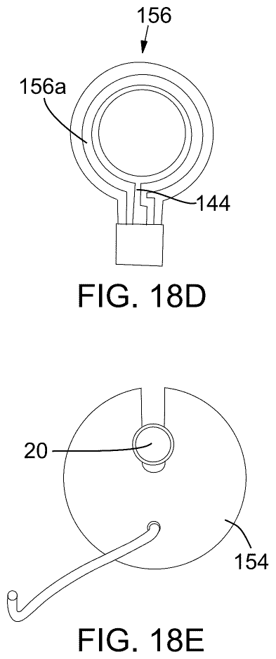

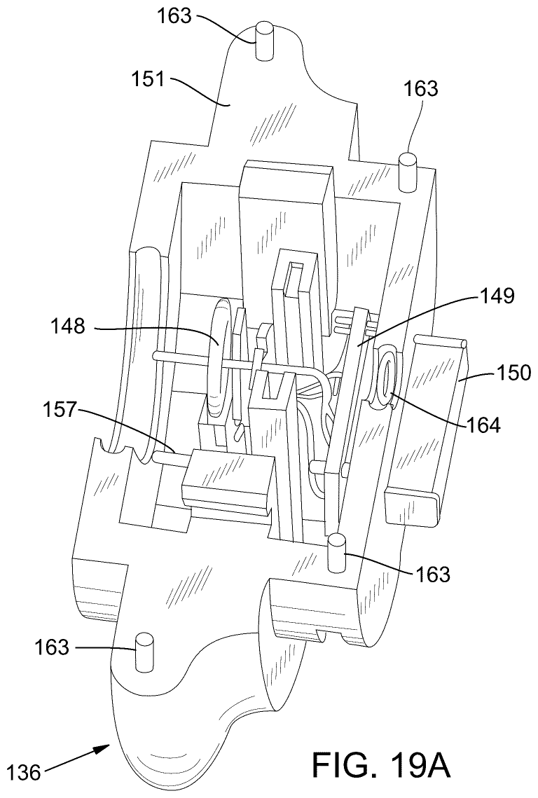

The distal rotating scope cavity 135 can be assembled over the distal end 22 of the metallic shaft 20, by sliding it (distal to proximal, dotted arrow in FIG. 17) to be secured at the proper longitudinal location along the length of the shaft. The exact location along the shaft where the rotating-element/scope cavity 135 is secured can be defined by a manufacturing jig to ensure that (a) when the cannula 95 is locked onto the distal end of 135, the length of the scope shaft 20 matches that of the cannula shaft, and (b) the orientation of the plane cut of the distal tip 22 of the scope shaft 20 aligns with that of the distal end portion 99 of the cannula shaft 95 (see FIG. 12F). An O-ring 100 can be applied to the distal end of 135 (FIG. 17) to prevent retrograde flow when gasses or liquids are infused through the cannula's luer port 98, when the cannula 95 is attached onto 135. Securing the distal rotating scope cavity 135 onto the shaft 20 can be achieved by either using an epoxy through the distal end 138 (FIG. 18B) or for example by utilizing two softer molded plastic pieces 145 that are inserted from the proximal end of the distal rotating scope cavity 135 and become wedged in place between the OD of the shaft 20 and the ID of a distal channel 158 of 135 (FIGS. 18A and 18B). Additional epoxy can be applied from the proximal and/or distal end of the component 135 to further secure all parts in place and seal from water ingress the inside cavity generated from the assembly of 135 and 136 around the shaft 20 of assembly 139.

For rotation detection and assessment, a potentiometer wiper 147 is attached onto the proximal end face of the distal rotating scope cavity 135 as shown in FIG. 18C. A wire is also shown coming off the wiper to further connect to electronics for measuring the resistance of the combined open potentiometer (156 and 147). This is the preferred rotation sensing transducer of the side-viewing scope in this embodiment. The conductive potentiometer wiper 147 shown in FIG. 18C is part of the innovation of this embodiment by utilizing a radial open potentiometer formed of printed polymer conductive paints such as the SENSOINK custom radial potentiometer models made by Hoffmann-Krippner (hoffmann-krippner.com/pet-sensoink/), see FIG. 18D for detail. Open potentiometers, such as the one suggested in FIG. 18D, are printed on FR4 or PET and require a conductive wiper such as 147. This can be a very low cost and reliable solution for a disposable product as described in this embodiment, where the conductive wiper 147 is contacting a conductive track 156a (that remains fixed inside 151) as the wiper gets rotated around (by rotation of 135). Thus, the wiper 147 and printable conductive ink track 156a together make the potentiometer in this embodiment. The layout of the wiper depends on the specific application, the resistor material, and design. Mostly so-called scoop wipers are used, but more and more they are replaced by scratch wipers. In any case the applications will determine the shape and material of the wiper, which can be customized for the specific use of the product (number of uses or mounting limitations etc).

Alternatively, the SENSOINK potentiometer design described above can be replaced by a radial SENSOFOIL membrane potentiometer (also custom made by Hoffmann-Krippner (hoffmann-krippner.com/sensofoil-membrane-potentiometers/). The shape can be very similar to that of FIG. 18D. Sensofoil membrane potentiometers consists of several layers, which are separated by a so-called “spacer.” The layers include: (a) A collector membrane as wiper tap for hand-, wiper- or magnet actuation; (b) Spacers between upper and lower membrane; (c) A basic membrane with potentiometer resistance track; (d) An adhesive film for attachment (depending to the application requirements). The membrane layers connect to each other through mechanical or magnetic pressure. The wiper 147 for a SENSOFOIL model of a printed radial potentiometer can be a totally passive non-conductive element such as a threaded screw with a soft rounded tip for example, or even a contactless wiper, if a magnetic Sensofoil model is chosen. Those knowledgeable in the art can readily utilize and mount a SENSOFOIL potentiometer track and wiper. The contact can be achieved either by hand or with a mechanical wiper. Contactless operation is also possible by using a magnet.

The printed potentiometer 156 can slide now over the proximal end of the fiber termination tube 140, and a grounding plate 154 (a metallic sheet-metal cut out) can snap onto the metallic shaft 20 as shown in FIG. 18C. A wire is also shown coming off the grounding plate for further connection to electrical ground. The grounding plate can be designed to mechanically flex and snap (by designed cutout features for example) onto the shaft 20 with a push over the shaft 20 as shown in FIG. 18E so as to make good mechanical and electrical contact with the metallic shaft 20. Such grounding can prove useful for ESD (electrostatic discharge) on the metallic shaft by quickly transferring such charge to ground and away from the imaging sensor, thus protecting the electronic imaging sensor at the distal tip 22 of the scope shaft from ESD discharges.

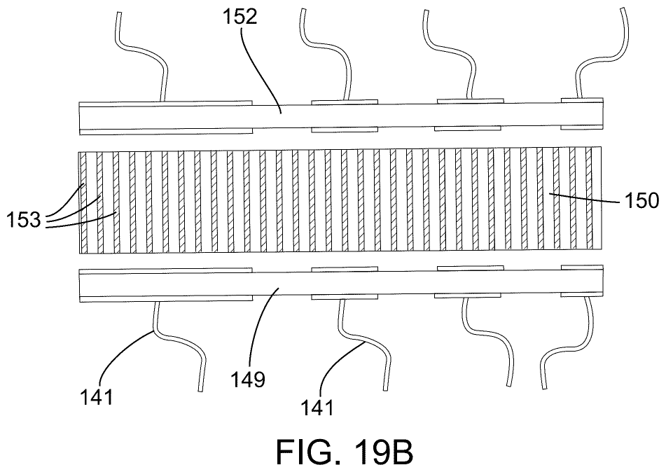

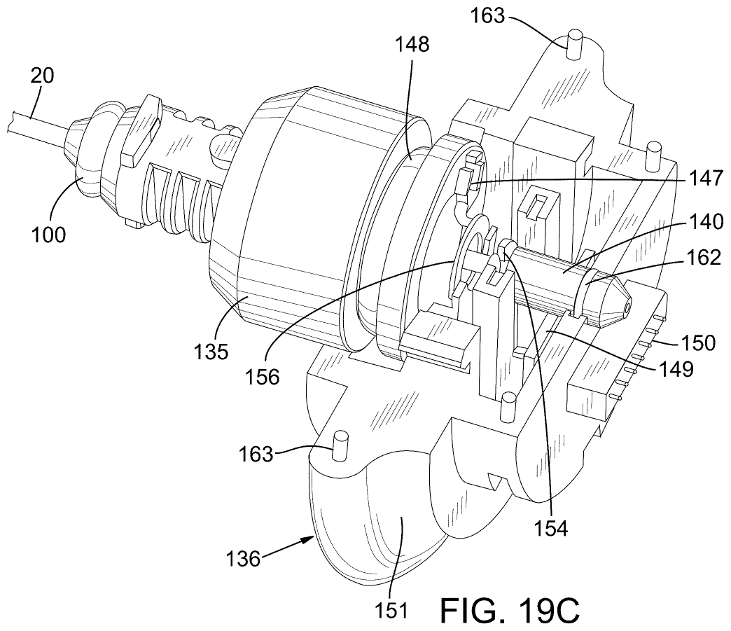

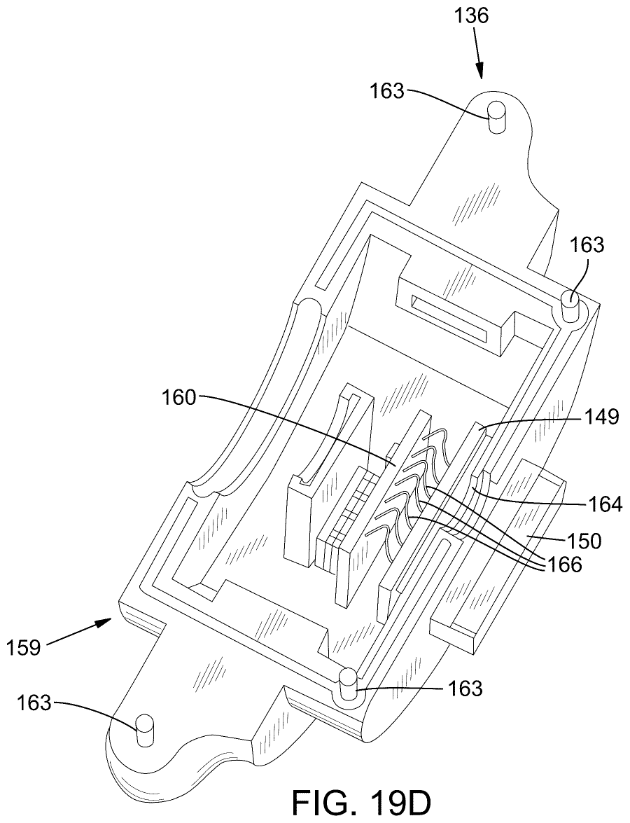

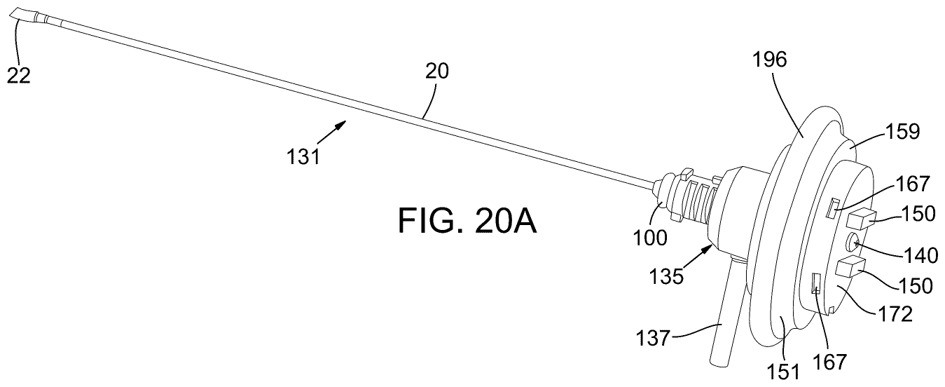

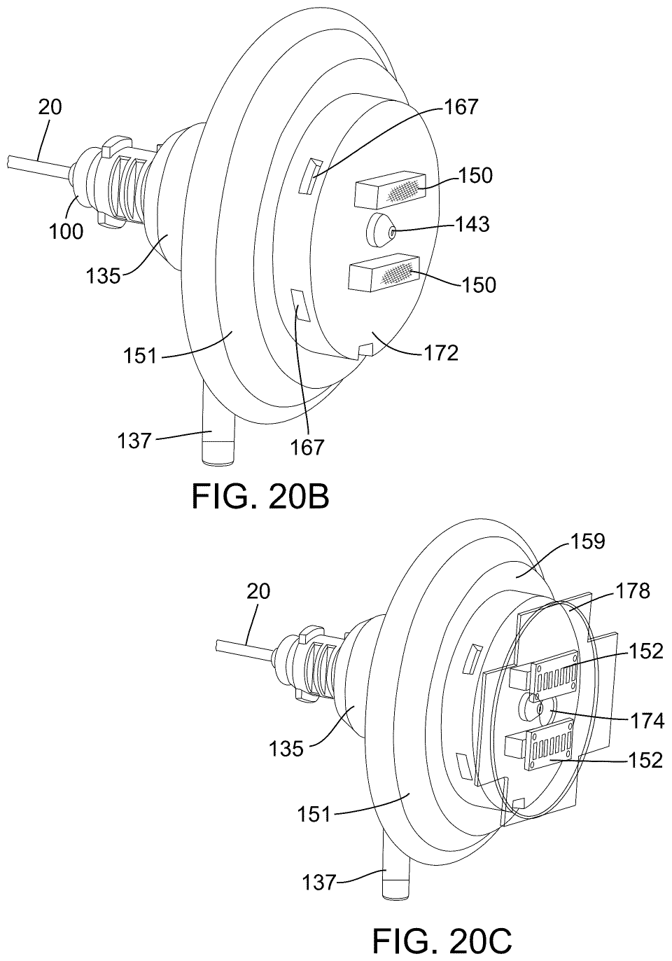

Before the subassembly of FIG. 18C can move on for further completion, the wires 141 from the sensor, the wire from the wiper 147, the wires from the potentiometer track 156a and the grounding plate 154 must first be soldered onto a passthrough PCB 149 shown in FIGS. 19A-D. An elastomeric connector 150, of well known configuration is then put in place through an opening of the bottom half 151 of the proximal fixed scope cavity component 136, and is assembled into contact with the PCB 149 (FIG. 19A is an exploded view). The passthrough PCB 149 is locked in place in the bottom half 151 as shown in FIG. 19A. The combination of the elastomeric connector 150 and passthrough PCB design 149 provides a reliable, low cost, and ingress protected electrical connection. The elastomeric connector 150 can be a custom design made by the Z-axis Co. such as the Z-wrap gold (zaxisconnector.com/products/z-wrap/) or other similar elastomeric connector designs. It consists of a soft elastomer with a multitude of gold conductive thin wires 153 equispaced and wrapped around it, as indicated in FIG. 19B, also showing the PCB 149 and a similar passthrough PCB 152 of the handle (discussed below), both to be pressed against the compressible connector 150. The spacing of the gold conductive wires 153 of the connector 152 can be less than 100 um so that multiple gold wires can contact the surface of even a small-width solder pad. The way the elastomer connector 150 and PCB 149 are used to make electrical connections with the reusable handle 132 is as follows: The passthrough PCB 152 (like the passthrough PCB 149 assembled in part 151) is positioned and aligned in the reusable handle 132 (FIG. 13) so that when the detachable scope 131 is connected to it, the solder pads of the two passthrough PCBs are completely aligned in space with one another as indicated in FIG. 19B. When the disposable scope 131 is connected to the reusable handle 132 and locked in place, the height of the elastomer connector, extending past the proximal end-face of the scope 131, is designed so that it can be slightly compressed between the two passthrough PCBs 149 and 152 while contacting both. See FIG. 20C. Those skilled in the art will easily recognize how to utilize such elastomeric connectors to make proper electrical connections of the signals between the detachable scope 131 and the reusable handle 132. Enough slack should be given to the wires soldered onto the passthrough PCB 149 in FIG. 19A, to allow for both ease of assembly as well as rotation of the rigidly connected assembly of 139 (FIG. 15) and 135 with respect to 136. Although any other style standard electrical connectors can also be used to communicate all the electrical signals from the detachable scope 131 to the reusable handle, including pogopin mating pairs, the elastomeric construct of FIG. 19B is the preferred embodiment for this invention, for its simplicity and low cost.

For limiting rotation a stop 146 can be included on the rotating scope cavity component 135 as shown in FIG. 18A, along with a mating stop 157 (FIG. 19A) on the bottom piece 151 of the fixed scope cavity 136, so that the rotation of the distal rotating scope cavity 135 is mechanically and rotationally aligned to avoid a dead zone 144 (FIG. 18D) of the SENSOLINK or SENSOFOIL track 138. In other words, in this embodiment, the rotation of 135 will be smaller than 360 degrees, which is a limitation of the SENSOINK and SENSOFOIL potentiometer track designs. The stops ensure that the wiper 147 (FIG. 18C) can scan the entire track 138 except for the dead zone 144.

At this point the sub-assembly of FIG. 18C can be placed onto the bottom piece 151 of the proximal fixed scope cavity component 136, FIG. 19C, by carefully placing the SENSOINK potentiometer 156 and the grounding plate 154 into the pockets that are specially designed in 151 to securely hold them in place. When the SENSOINK potentiometer 156 is securely held in place, the wiper 147 is designed to make electrical contact with its track 156a, FIGS. 19C and 19D. The O-ring 148 near the proximal end of 135 will further protect against liquid ingress across the interface between the distal rotating scope cavity 135 and the proximal fixed scope cavity 136, while allowing for rotation of part 135 against parts 151 and 159, when assembled, as seen in FIG. 20A.

Before this assembly the top half 159 of the proximal fixed scope cavity 136 is prepared, as shown in FIG. 19D. In this half, a miniature PCB 160 can be secured in place that contains a Non-Volatile memory (NVRam) chip that can be programmed during manufacturing to hold information about the specific disposable assembly, such as model and lot number, rotational and other hardware calibration data, and calibration data of the imaging sensor (among other data). Electrical wire connections 166 between the NVRam chip 160 and a second passthrough PCB 149 (similar to that in FIGS. 19A and 19B) can be made first. Then a second elastomeric connector 150 is passed through the opening on the proximal end of 159 and secured in place from behind with the second passthrough PCB 149. The NVRam chip PCB can be replaced by a RF ID tag inside the top half 159 that can communicate such calibration and product info wirelessly to a corresponding receiver inside the reusable handle 132.

With these components in place the top half 159 is ready to be locked in place onto the bottom half 151 and around the rotational component 135. Mating alignment and locking features 163 can be designed onto the molded pieces 151 and 159 for this purpose so they can snap together without the need of epoxy or screws; see FIGS. 19C and 19D. An appropriate form of gasketing 165 or even a mechanically designed seal can be applied on the perimeter of upper half 159 (shown in heavy line in FIG. 19D) to further seal the interface between 151 and 159 from water ingress.

An additional O-ring 162 over the fiber termination tube 140, shown in FIG. 19C, can provide water ingress protection of the opening 164 (FIG. 19A), through which the fiber termination tube must extend out of the proximal end of the fixed scope cavity 136 as seen in FIG. 19C. The O-ring 162 and the opening 164 it seals must be designed so that the tube 140 can rotate with little friction while the O-ring 162 can still protect against water ingress.

Thus, the sealing of the distal end 138 of the rotatable component 135, the O-ring 148 at the proximal end of 135, the gasket 165 on the perimeter of the top half 159 and the O-ring 162 on the fiber termination tube 140 can together provide complete protection against water ingress of the inside cavity of the detachable scope defined by 135, 151 and 159.

To be commensurate with the manner of construction of standard arthroscopes, it is preferable for the removable lever 137 (FIGS. 13A and 13B) to be mounted onto the distal rotating scope cavity 135 opposite the direction that the side-viewing plane of the distal end-face of the scope 22 is facing. Thus, if the sideviewing plane is rotated to look at 12 O'clock the removable lever 137 should be pointing at 6 O'clock; see FIG. 20A. It is also preferable for the rotation lever 137 to extend past the OD of the annular rim 196 of the fixed component 136 so that the user can access it by reaching over the ridge 196 with a finger, while holding onto the handle 132. See FIGS. 13, 20A and 23.

The fully assembled disposable/detachable scope 131 is depicted in several views in 20A through 20C, ready to be attached to the reuseable handle 132. The proximal sides of the assembled lower and upper halves 151 and 159 are designed with locking features 167 to ensure (a) proper alignment of the passthrough PCBs 149 on the detachable scope 131 with the corresponding identical passthrough PCBs 152 on the reusable handle, (b) proper alignment of the center of the proximal end 143 of the fiber termination tube 140 with the optical output 174 of an LED emitter that resides inside the reusable handle, and (c) mechanical locking of the scope 131 onto the reusable handle 132. FIG. 20C, depicting the position of the distal end-face 178 of the reusable handle 132 in transparent mode, with respect to the proximal end face of the disposable scope 131 when the two assemblies (131 and 132) are locked in place, provides further clarity on the necessary alignment of the passthrough PCBs between the two assemblies (as pointed out in FIG. 19B). FIGS. 20B and 20C also highlight the necessary alignment of the proximal end face 143 of the fiber termination tube and the optical output 174 of the LED emitter that resides inside the handle, to ensure sufficient optical transmission to the illumination fibers that run from the proximal end-face of the fiber termination tube 140 to the distal end 22 of the shaft.

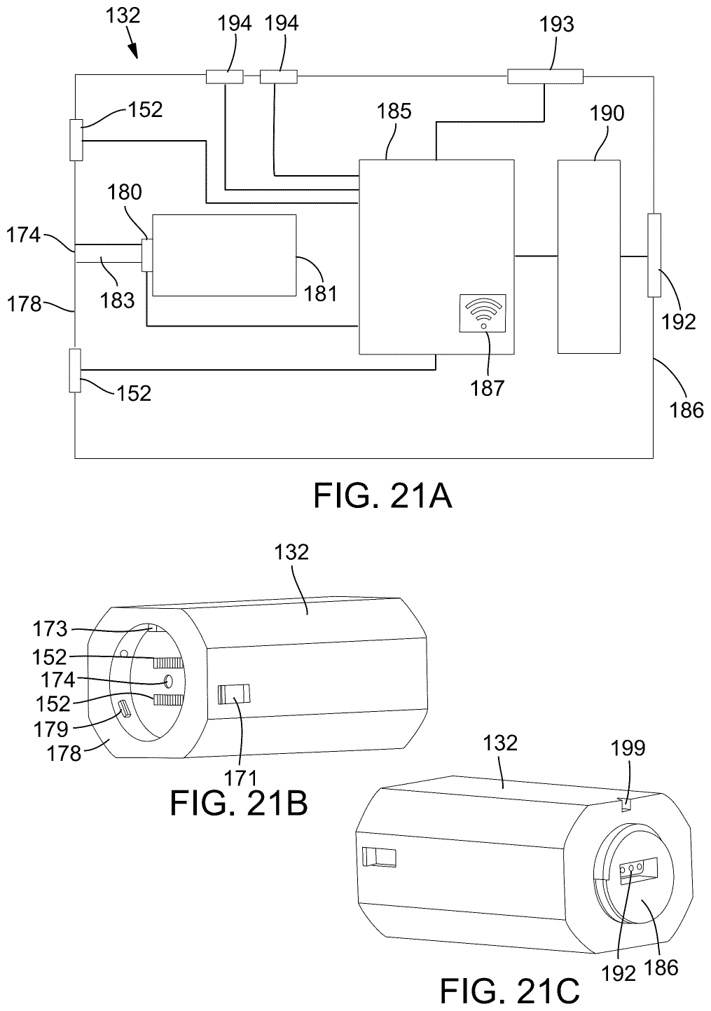

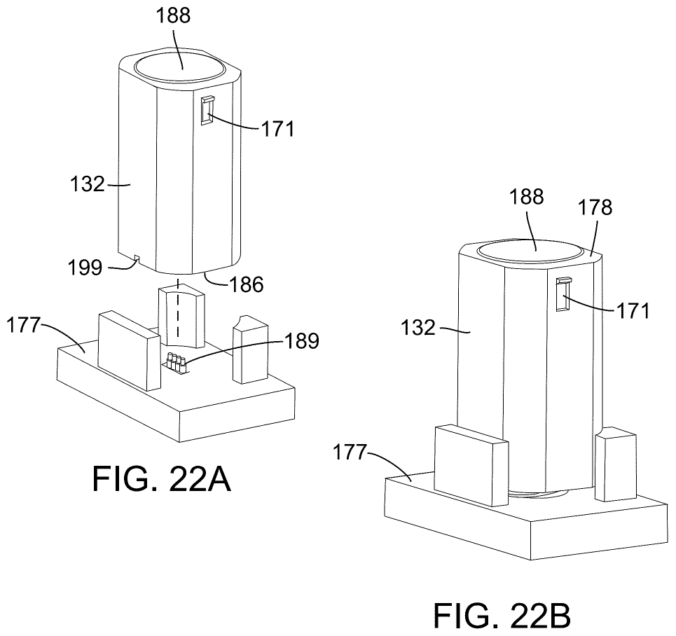

To ensure that the scope can only be locked with the reusable handle 132 in a specific rotational orientation, asymmetric features 172 (on the scope 131, FIG. 20B) that can mate with corresponding asymmetric features 173 (on the handle 132, FIG. 21B) in only one orientation are built on the interface between the two pieces. Once the scope 131 is locked in place against the reusable handle 132, it can be disconnected by pressing or sliding on a lever 171 that releases handle latches 179 (FIG. 21B) and unlocks them from locking features 167 on the scope 131, and thus releases the scope 131 from the handle 132.

A block diagram indicating preferred contents of the reusable handle 132 is shown in FIG. 21A.

The distal end 178 of the reusable handle 132 mates with the disposable scope 131. The proximal end 186 of the handle in this rechargeable embodiment mates with a docking station 177 (FIG. 22) when charging. The light source that provides illumination for the scope is preferably an LED 180 (FIG. 21A) that is mounted to a heat sink 181 for thermal management. Light out of the LED is captured and guided with a light guide 183 onto a plane 174 that matches the location of the proximal end face 143 of the fiber termination tube 140 (FIG. 20) for effective optical coupling. Such light management can be achieved by either a TIR (Total Internal Reflection) lens, or a light pipe, or by moving the LED emitter as close as possible to the proximal end face 143 of the fiber termination tube 140 when the scope 131 is locked in place with the handle 132. Those skilled in the art of non-imaging optics can design such transmission and light coupling system for efficient light coupling from the LED emitter to the fibers that reside within the length of the shaft 20 of the disposable scope 131 and transfer light to the distal end 22 of the shaft for proper imaging. In the preferred embodiment of a cordless handle 132, rechargeable batteries 190 are also housed inside the handle 132 and are electrically connected to an IP rated connector 192 for contact charging with the docking/charging station 177 of FIG. 22. Inductive charging can also be used, in which case the connector 192 would not be needed. If the handle is wired to the image processing hardware 11, 190 or 192 are not needed. Instead, a multiconductor cable 14 (FIG. 1), can communicate all electrical information to the image processing hardware outside the handle, while also supplying power to the scope. Such cable preferably exits the proximal end 186 of the handle such as shown in FIG. 2. Lighted indicators 193 at the outside of the handle can be used to indicate the charging state of the batteries, if included, as well as the wireless connectivity strength with the image processing hardware, or any other information that may need to be communicated to the user. A multitude of IP rated buttons 194 can also be on the exterior for different functionalities, for example to snap pictures, or to start/stop video capture, and to turn power on for the wireless reusable handle 132.

A PCB 185 inside the reusable handle will manage all the electrical and electronic functions such as:
Manage the NVram data from the disposable scope 131. In the case of a RF ID tag, then the handle PCB shall contain a corresponding reader to access such information from the disposable scope.
Communicate with the image sensor at the distal tip 22 of the shaft 20 of the disposable scope 131.
Communicate with the rotation transducer 156 from inside the disposable scope 131, as well as manage the ESD protection grounding plate from the disposable handle.
Drive the LED.
Manage the buttons and the lighted indicators and transfer such information to the tablet or monitor outside the handle.
Perform all necessary power management of the rechargeable batteries.
Perform some preliminary image processing and prepare the image data from the sensor for transfer to the image processing hardware outside the handle (either via wired or wireless communication).
In the embodiment of a cordless handle, drive and manage a wife transceiver 187, that resides inside the handle for communicating all info with the image processing hardware and tablet.

The enclosure of the reusable handle is preferred to be designed with some level of Ingress Protection (IP) against water ingress, preferably IPx4 that protects against sprays from a broad range of angles. Other IP ratings can be implemented depending on the intended use of the scope.

The reusable scope 132 can be wired to the video processing hardware 11 via a cable 14 (FIG. 1). In a preferred embodiment, the handle 132 is communicating with the video processing hardware 11 wirelessly. In such embodiment the handle 132 must contain batteries 190 that can be re-charged on a charging docking station 177 when not used, as in FIGS. 22A and 22B. The docking station can charge the handle inductively (no physical connection), or by contact charging. If contact charging, it is preferred to mount the handle 132 with its proximal end 186 against the charger, as in FIG. 22B. In this arrangement it is preferred to add a dust cap 188 over the distal end 178 of the handle to protect the electro-optical connections from gathering dust.

With contact charging, it is preferred to use a water ingress protected mating pair of pogo pins (192 on the handle and 189 on the docking station) that are installed at proper locations on the docking station 177 (FIG. 22) and on the proximal end of the reusable handle 132 (FIG. 21C), so that when the handle rests in the docking station, proper electrical contact can be made between the two pairs of pogopins. IP rated Pogopin pairs made by Mill-Max corporation (or others) work well (mill-max.com/products/new/spring-loaded-pogo-pins) for this embodiment. Asymmetric mechanical features 199, on the interface between the docking station and the proximal end 186 of the reusable handle 132, can be designed to ensure a unidirectional docking and charging of the handle, FIG. 22A. The charging station 177 can also have lighted indicators for indicating the charging state of the batteries in the handle 132.

The embodiment of 130 (131 and 132 together) can be used clinically from as simple a setting as a doctor's office to as complex a setting as a surgical suite. But on any occasion, since the handle 131 is reusable, it will need to be reprocessed or re-sterilized before every use. Depending on the clinical setting, the reusable handle can be designed to be re-sterilized by autoclaving it or some other sterilization process such as ethylene oxide or oxygen peroxide sterilization cycles. At the same time, it can also be designed to be reprocessed by a chemical process of wiping its outside surfaces with proper disinfectants, and/or soaking it in appropriate disinfection agents. Those skilled in the art of reprocessing can easily design hardware and electro-opto-mechanical assemblies (such as that of the reusable handle 132) to withstand multiple cycles of re-sterilization or re-processing.

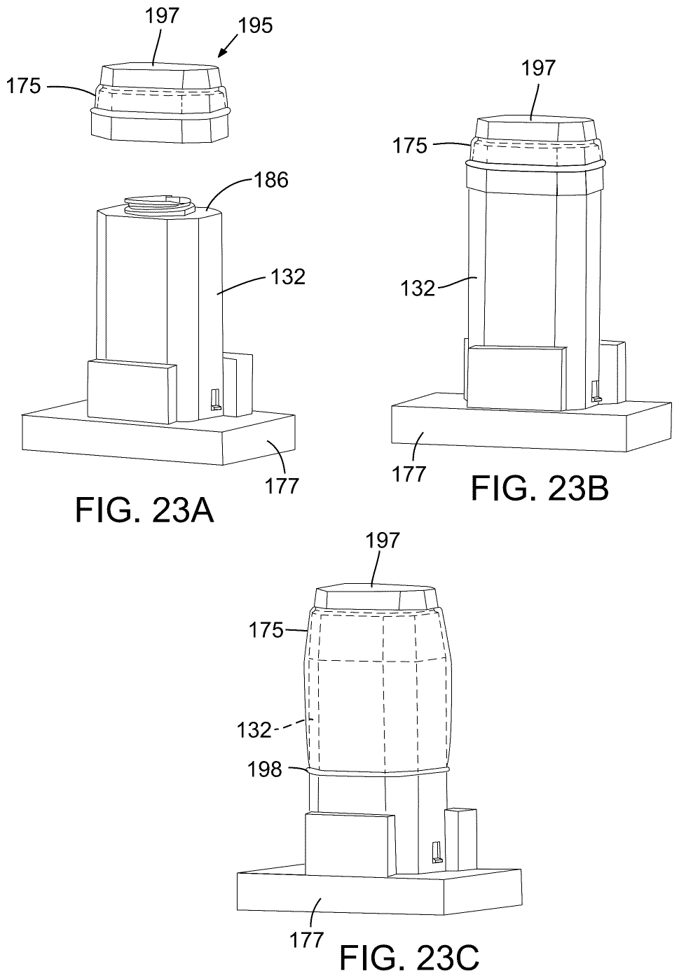

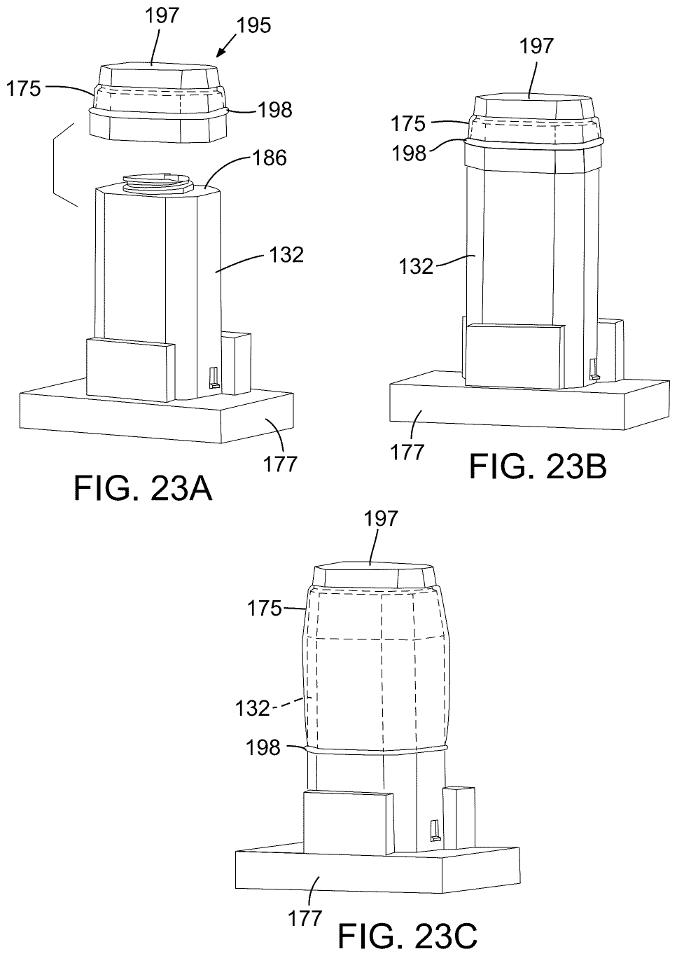

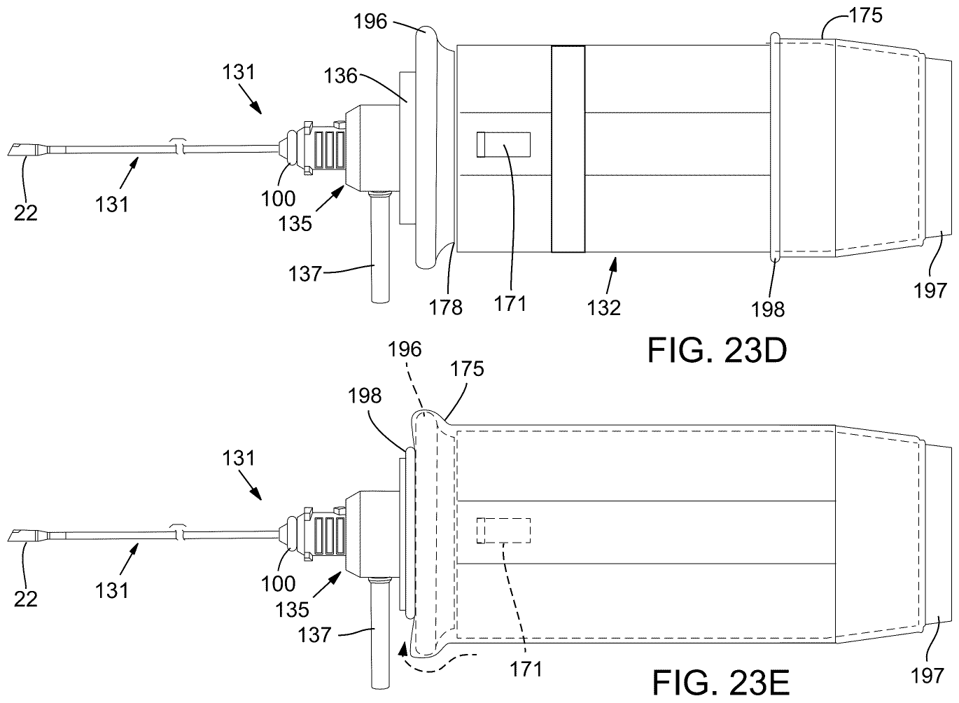

Another preferred embodiment of 130 is one where neither a re-sterilization nor reprocessing is required for reusing the non-sterile handle 131. Instead, according to the invention a specially designed sterile sheath 175, as in FIG. 23A-C, can be applied over the distal end of the non-sterile handle 132, in a simple fashion, by using only one hand wearing a sterile glove. This way the reusable (non-sterile) handle 132, under the cover of a sterile sheath 175, can be connected to the sterile single use scope 131 and brought in the sterile environment of an operating theater for use in an endoscopic procedure.

Unlike some prior art, such as U.S. Pat. No. 8,317,689 (and instructions for use of the Visionscope system) where the application of a sterile sheath over a reusable scope assembly is cumbersome and requires the user to wear multiple sterile gloves and use both hands, it is the purpose of this invention to provide a sterile sheath and its application onto a reusable scope handle by a single hand operation using only one glove.

To apply the sterile sheath 175 over the reusable unsterile handle 132 with one hand, it is preferable that the handle is resting on the charger with its distal end 178 against the charger, FIG. 23A (opposite of the charging orientation depicted in FIGS. 22A and 22B). Thus, the user, before donning sterile gloves, must first flip the handle from its charging orientation of FIG. 22B (after removing the dust cover 188) to the position depicted in FIG. 23A. With the handle 132 resting in the charger as in FIG. 23A, the user can now wear one set of sterile gloves. The sterile sheath assembly 195 of this embodiment in FIG. 23 consists of two components: (a) the holder 197, and (b) the sterile sheath 175 itself. The sterile sheath 175 is attached to the holder 197 and is rolled up against it, FIG. 23A. The sheath assembly 195 is preferably packaged with the single use scope 131 in a tray that is sterilized beforehand. The cap 197 has features or threads that can easily mechanically lock and unlock it onto the proximal end 186 of the handle 132.

The user, wearing sterile gloves, can reach into the sterile tray and pick up the sterile sheath assembly 195 with one hand, then lock the cap 197 onto the proximal end 186 of the handle 132 (FIG. 23B), and proceed to roll the sheath down over the handle to a point where enough of the body of the handle is covered by the sheath so that it can be picked up off the docking station 177 with one hand by holding onto the covered portion of the handle, FIG. 23C. Then the user with the other hand (also sterile and gloved) reaches into the sterile tray and picks up the sterile scope 131 and connects it onto the distal end 178 of the unsterile handle by simply pushing the sterile scope 131 (in the proper orientation) into the distal end 178 of the reusable handle 132, as in FIG. 23D. Then by holding with one hand more firmly on the sterile scope side of the connected assembly (of the scope and handle), the user can use the other hand (that is holding onto the handle at the portion that is covered by the sheath 175) to further roll the end-rubber 198 of the sterile sheath farther distally on the handle and to push it over the round annular ridge 196 of the sterile scope, as in FIG. 23E. At this point, by wearing only one set of gloves on each hand, the user has applied a sterile sheath assembly 195 over the whole body of the non-sterile reusable handle 132, and locked the sterile sheath 175 against the sterile scope so that the sheath cannot come off during use of the completed assembly 130. Thus, in essence a sterile barrier has been applied completely over the reusable, non-sterile scope, as in FIG. 23E.

As part of this embodiment, it is the intent of this invention for the ridge 196 of the fixed portion of the scope 136 to be slightly larger in size than the width of the handle and the nominal size of the end-rubber 198. At the same time, the elasticity of the end-rubber 198 is such that it can be pushed and rolled with a small force over the ridge 196, so that it can remain in place as in FIG. 23E during use and prevent the sterile sheath 175 from rolling back out over the ridge 196 exposing the body of the non-sterile handle 132. The sterile sheath 175 is preferably constructed of a thin transparent plastic so that features of the handle 132 that need to be visible to the user (such as the lighted indicators or buttons) can continue to be visible through the sheath.

At the end of the procedure, the user can roll the end-rubber sheath 198 back out and over the ridge 196, then roll the sheath 175 further out of the handle, unlock the cap 197 and discard the whole sheath assembly 195. Then by pressing or sliding the lever 171 (FIG. 21B), the user can unlock the used scope and discard it as well. At this point the reusable handle is free and can be placed back onto the charger 177 as in FIG. 22E to charge the batteries and be ready for the next procedure.

In another embodiment of the sterile cover over the wireless non-sterile reusable handle 131, the sterile sheath assembly 195 can be made entirely of a harder plastic cover piece (or more than one piece) that can completely encapsulate the reusable scope handle 132. In this case, the assembly 195 would be more of a sterile cover than a sterile sheath. Such cover assembly would clip over the reusable handle and would reach out distally and attach onto features such as the ridge 196 or some other feature on the on the fixed portion 136 of the disposable scope 131. Transparent conforming windows on such plastic sterile cover would allow access to the buttons (for mechanical actuation) and the indicators light (for viewing) through the windows.

In the case the reusable handle 132 is wired to the image processing hardware, then the sterile sheath assembly 195 can be applied from the opposite direction on the handle (distal end roll over the handle proximally). In this case the docking station 177 can be just a passive mechanical holder that can hold the handle 132 in an orientation similar to that depicted in FIG. 22B (with the distal end 178 of the handle 132 facing upward) while allowing for the electrical cable coming out of the proximal end 186 of the handle to get through the docking station (while its distal end 178 is upward). In this embodiment the sterile sheath assembly 195 can be slightly different from that depicted in FIGS. 23A-23E, with its holder 197 already attached onto the fixed portion 136 of the sterile scope and with the end-rubber 198 of the sheath assembly facing toward the proximal end of the sterile scope 131.

The user can then take the sterile scope with one hand (wearing a sterile glove) and lock it onto the proximal end 178 of the handle by pushing it onto the distal end of the handle until it locks in place (without touching the unsterile handle). Then the sheath 175 is rolled over the handle until enough of its length is covered by the sheath so the handle can be picked up off of the holder 177 with one hand. Then the other hand, also gloved with a sterile glove, can continue to roll the sheath 175 out toward the proximal end 186 of the handle until the whole handle 132 is covered. In this embodiment the user will need to continue to unroll the sterile sheath over the entire length of the electrical cable 14 that is connected to the handle's proximal end all the way to the image processing connection at 11.

In a previous embodiment the orientation of the wireless reusable scope on the docking station 177 while charging was with its proximal end against the charger. Thus the charging pins 192 were located on the proximal end 186 of the handle. The reason is that the distal end 178 of the handle contains already several electro-optical interconnects with the disposable scope 131, and other locking and mating features 179, 173 with the scope. The location of the charging pins on that end could make the assembly more cumbersome, but it is possible to provide for charging the handle 132 on its distal end.

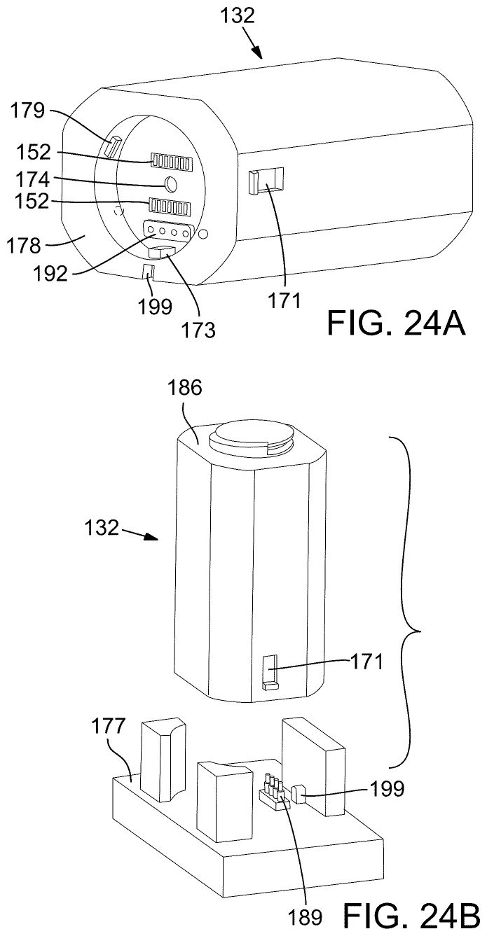

In this embodiment, depicted in FIGS. 24A and 24B, the charging pogopins 192 reside on the distal end 178 of the reusable 132 as depicted in FIG. 24A, again in the proper location so that when the handle 132 is resting in the docking station 177, pogopins 192 make proper contact with their mating counterpart 189 on the docking station, as in FIG. 24B. Latches 179 that lock onto the scope, and asymmetric features 173 to ensure proper orientation of the scope, still are present on the distal end 178 of the reusable handle, along with passthrough PCBs 152 and the optical output window 174. In this embodiment, with the scope charging facing down, there is no need for a dust cover while charging as was the case with the configuration depicted in FIG. 22B. Since the scope is charging at the same orientation as that of FIG. 23A, the application of the sterile sheath assembly 197 will be identical to the previous description. Clearly for this embodiment, the location of connector 192 in FIGS. 21A and C will have to move to the distal end 178 of the handle.

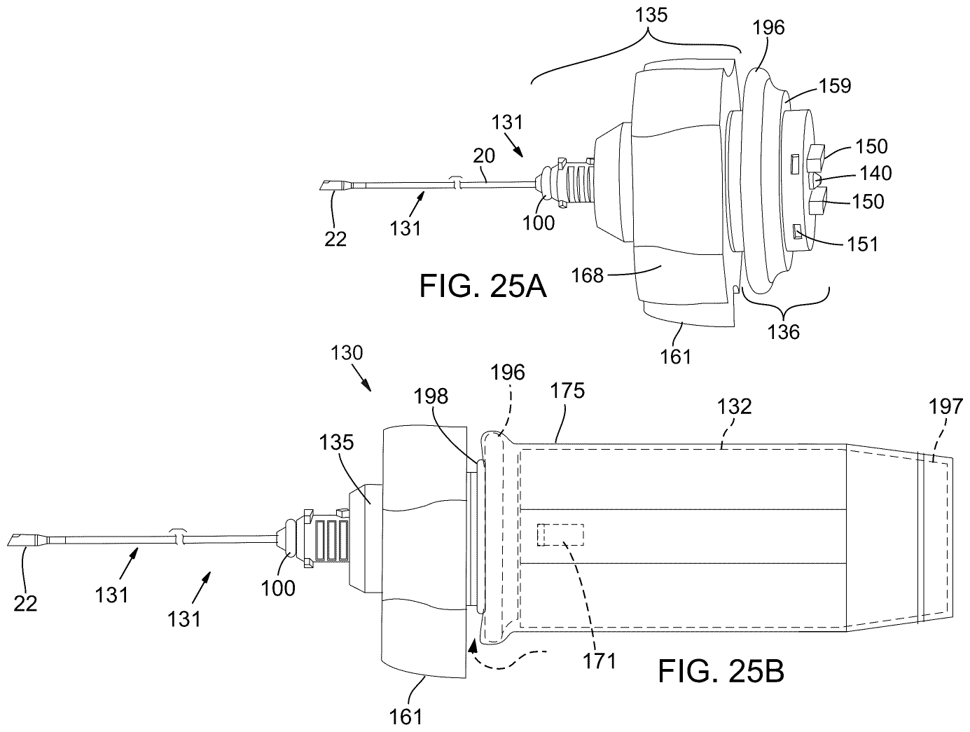

In yet another embodiment shown in FIGS. 25A and B, the removable lever 137 in FIGS. 13 and 14 can be replaced with a circumferentially oversized feature 168 on the distal rotating scope cavity 135. In this embodiment of the distal rotating scope cavity 135, the outside surface of the circumferential feature 168 can appear similar to that of the outside surface of part 36 in FIG. 4 for example. The oversized feature 168 should be preferably larger in diameter than the OD of the annular ridge 196 (see FIG. 25A) so that the user can easily access the feature 168 with one hand over the ridge 196 (while holding onto the handle) and rotate 135. An additional asymmetry on the outer surface 168 of 135 can be added in the shape of a small shark fin 161. Shark fin 161 is located radially at the same location with respect to the viewing angle of the scope as was the removable lever 137, i.e. diametrically opposed orientations. This way the user can feel with fingers (by the touch of the shark fin 161) the orientation of the sideviewing without having to look down on the scope. All other features in this embodiment can be the same as the embodiment shown in FIG. 20A.

With this embodiment a sterile sheath can be used as described previously.

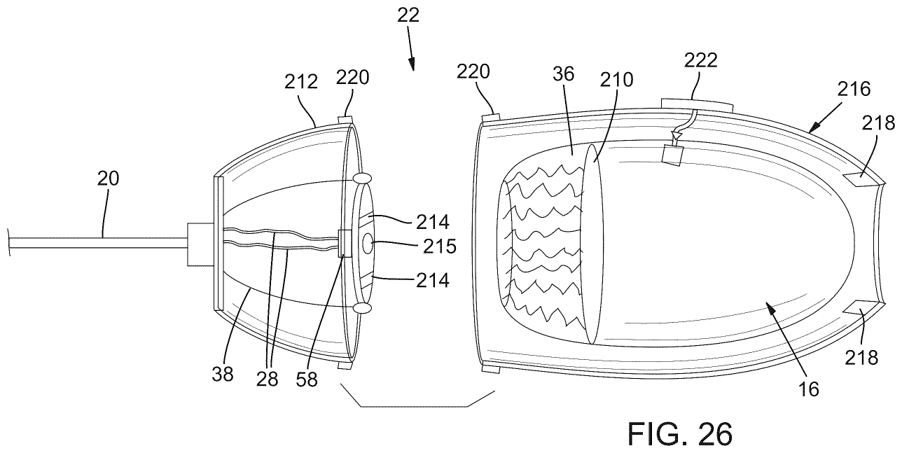

FIGS. 26 to 28 show another embodiment for sterile protection of the instrument of the invention in use. In FIG. 26, a schematic cross sectional view, the distal end of the instrument, including the shaft 20 and shaft-retaining component or needle base 38, is disposable and separable from the handle 16, 36. The portion 36 of the handle is rotatable, at the indicated rotation plane 210. At the distal end, a plastic hood or shell 212 is fixed to the shaft retaining component 38 and rotatable therewith. The shaft retaining component 38 includes an LED 58, with optical fibers indicated at 28 in the drawing. Electrical contacts are indicated at 214 and a heat sink transfer pad at 215.

On the right side of the drawing, the handle 16, 36 (albeit with a wireless version of electronics included in this handle) resides within a proximal hood or shell 216. The handle 16, 36 (wireless) is engaged within the proximal shell 216, such as by frictional or snap-in or twist-and-lock engagement. Orientation of the handle's rotate portion 36 with respect to the proximal shell is defined by pins or lead-in features or other features. Snap-in clips are indicated in the proximal shaft at 218 in the drawing. This is so the handle can be retained within and removed from the proximal shell 216. Other forms of clips or frictional engagement can be used.

As noted above, rotation of the cone piece 36 is indicated at the rotation plane 210 in the drawing. In addition, the distal shell or hood 212, when snapped together with the proximal hood or shell 216, via snap tabs or fittings 220 on each, preferably provides for rotation of the distal shell 212 (with the shaft retainer piece 38) relative to the proximal shell 216. However, the two shells can be fixed without relative rotation, but with the needle and shaft retainer piece 38 rotatable (with the cone piece 36). A radial lever (not shown) can be included on the exposed needle hub for this purpose.

However, prior to connection of the two shell pieces together, cone piece 36 of the handle is first snapped together with the shaft retaining component 38. This can be via connections discussed above, and provides for electrical connections to be made between the two sides via the electrical and heat contacts 214 and 215 and similar contacts on the cone piece 36. The proximal shell 216 is snapped onto the distal shell 212 after that internal connection has been made.

FIG. 26 indicates an on/off switch 222 on the outside of the proximal shell 216, for making appropriate contact with a conductor on the handle to operate the device via the outer shell 216. When all the components have been secured, the needle 20 can be rotated by the user, by manually turning the distal hood or shell 212 relative to the proximal shell 216, and this will effect rotation at the rotation plane 210 in the handle.

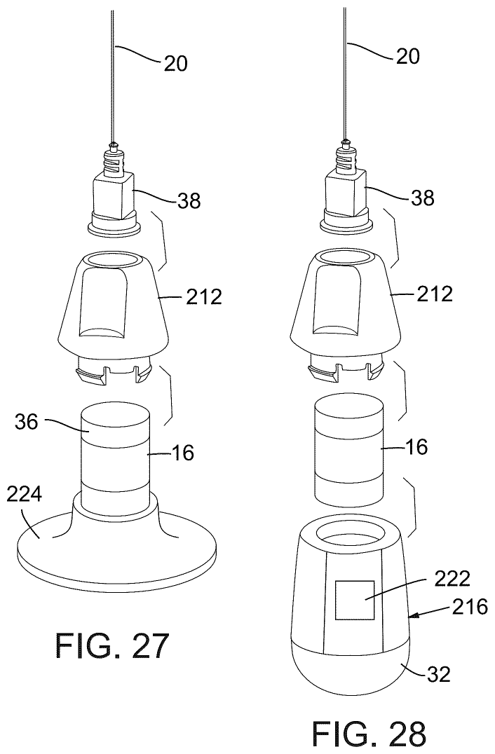

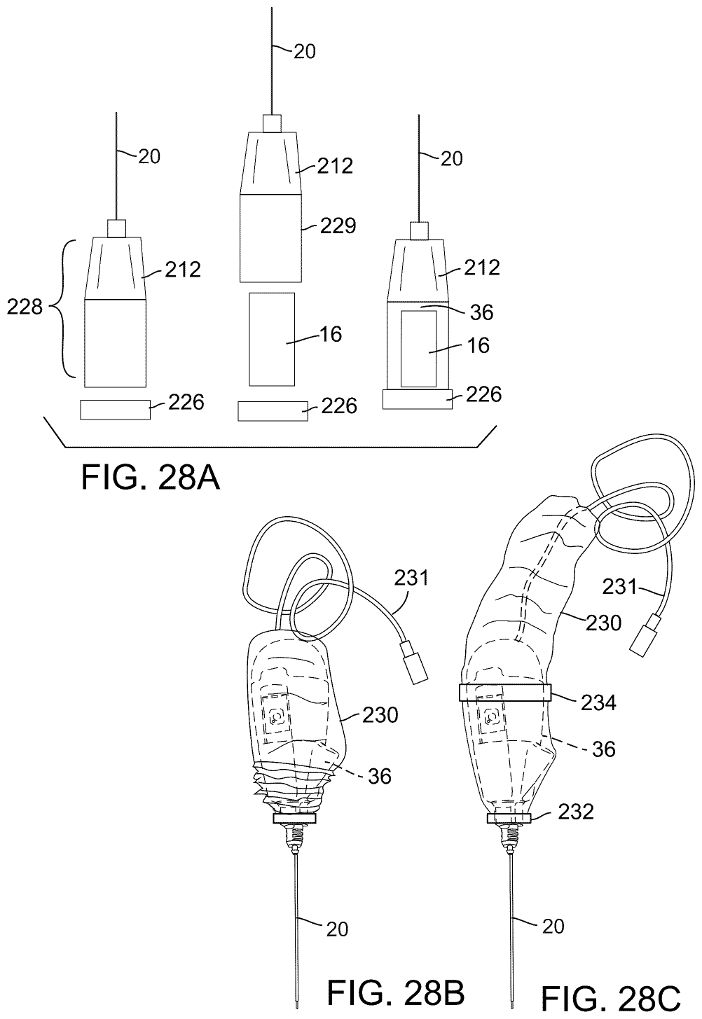

FIGS. 27 and 28 further explain the sterile use of the device. In FIG. 27 the handle 16, 36 is docked in a charge stand 224. The drawing indicates the shaft retaining piece, retained within the distal outer shell 212, being lowered onto what is seen as the upper end (distal end) of the handle, to be assembled together with a snapping attachment or a “click”. The outer shell 212 and needle 220 are sterile, having been removed from a sterile wrapper or container.

Then, the assembly of the distal outer shell 212 with the handle secured to the shaft retaining piece 38 is lifted off the charging station 224 and is lowered into place on the proximal outer shell 216, as indicated, which inserts the handle body 16 down into the gripping mechanism 218 of the shell 216. Like the distal shell 212, the proximal shell has been sterile and unpacked from a wrapping. Again, the outer shells 212 and 216 are snapped together with a “click”, a connection that can be released later. In this way, the sterile needle shaft 20 and sterile outer shells 212 and 216 are unpacked, secured to the reusable handle 16, and connected together in rotatable fashion with the reusable handle 16, 36 contained inside. This avoids any need for sterilization of the reusable handle. At the conclusion of a procedure using the videoscope of the invention, the shells are separated from each other by a quick release device, leaving the distal shell 212 and handle 16, 36 essentially as seen in the upper part of FIG. 28. The snap-together connection between the needle retaining piece 38 and the cone piece 36 (FIG. 26) is then accessible to the user, and separation of the components 38 and 36 (i.e. separation of piece 38 from the handle) can be accomplished by pushing an appropriate final release mechanism (not shown). The components on the left side of FIG. 26, as well as the proximal outer shell 216, are discarded, while the handle 16, 36 is retained and reusable.

At the end of a procedure a logical and easy process of removing the disposable shells is provided, such as removing the back (proximal 216) disposable first via simply applying force by hand. Then the front (distal 212) disposable and reusable handle remain in contact, so in the following step either via the release mechanism discussed or by force the front disposable (shell 212 with piece 38) is disengaged and discarded. In a different embodiment indicated systematically in FIG. 28A, the back disposable is simply a cap 226 that can be rotated or snapped onto a much larger front disposable piece 228 that could also include the on/off and operating buttons. This will involve sliding the reusable handle 16, 36 into that longer front disposable 228 and then simply adding the back cap 226 that attaches to the back of a main back shaft body 229, the latter being rotatably connected at its front (distal) end to the front (distal) shaft 212. Either the back cap 226 or the main back shaft body 229 attaches to the reusable handle portion 16. The front shell with needle 20 is rotatable relative to the back shell 229 and cap 226. This construction keeps the reusable intact and secure has advantages of ease of use.

In one embodiment of the invention the portable endoscope instrument is cordless, with a rechargeable battery and wireless connection from the instrument, including image data, to the image processor and monitor. The protocol for video and general data transmission from the portable endoscope to a base unit can be WiFi or Bluetooth moving compressed or uncompressed video data or point-to-point data transfer such as direct WiFi. The size of the portable endoscope enables it to be easily handheld. Also, the scope can be used with single-hand operation and manipulation. The base unit (11 and 12 in FIG. 1) can be specifically built-in hardware and firmware and software, such as an embedded Linux industrial level tablet, or can be a consumer grade iOS operating system tablet from Apple Corporation, Cupertino, California or an Android tablet formed Samsung Corporation of Korea. The tablet and/or the handheld can, if desired also communicate with a separate display monitor or multiple displays displaying video, images, data or other relevant information.

One embodiment/example (not shown) is for the heat sink for the LED to also create a support member of the handle. The LED and heat sink are interfaced in a constrained manner with the front tip of the endoscope which can be in the form of a long needle with the camera at the distal end while the heat sink is at the proximal end. The rotation-detecting potentiometer, which can be in the form of a knob-rotating potentiometer with a non-rotating base, is mounted on the proximal end of the heat sink. The base of the potentiometer can be fixed with respect to the heat sink, which can be cylindrical (as at 44 in FIGS. 5 and 5A), and so the knob is on the long axis of the heat sink cylinder and pointing toward the proximal end of the full handle, i.e. toward the operator, while the patient is at the very distal end of this axis. The front, rotating member of the handle is then comprised of the camera with optic, tip, needle, fibers, LED, heat sink and potentiometer. In that case the back (non-rotating) portion of the handle is mounted on the potentiometer/rheostat knob to provide for relative rotation between the knob and the front rotating member of the handle. In another example the potentiometer with knob can be flipped and the knob can be attached with appropriate adaptor onto the proximal end of the heat sink (which is in the front rotatable part) while the potentiometer base is now attached to the back portion of the handle. The front and back portions rotate with respect to each other, effecting rotation between the two parts of the potentiometer. Depending on the choice of the configurations described above, the wires connecting the potentiometer base that provide voltage and measure the voltage that correlates with rotation angle are connected to the PCB board and can be twisting while under rotation. Thus the wires could also provide a rotation limit of either side of the rotation with respect to, for example, a location of a button on the handle or other feature. More preferably a separate feature limiting the rotation can be used and the wires can be longer, never drawn taut. One example of such a potentiometer can be PN 5A1A-B28-A15L from Bourns Inc. and sold from Digikey Inc. website with a description of: “10k Ohm Gang Linear Panel Mount Potentiometer None Kierros Cermet 2 W Solder Lug”.

Another embodiment would be to have a potentiometer with a hole instead of one with a knob. FIGS. 5 and 5A show such an embodiment with potentiometer 50 having a hole where a feature of the heat sink or a separate adaptor makes contact through the hole with the potentiometer and allows for rotating the potentiometer inner cylindrical body with respect to the potentiometer outer body that is fixed. Such a potentiometer can be, for example, part number PN 3382H-1-103 from Bourns Inc and sold from Digikey Inc. website with a description of: “Resistive Sensor Rotary Position Hole for Shaft PC Pin”.

FIGS. 28B and 28C indicate another embodiment that introduces a disposable sterile barrier 230 over both rotating and fixed portions of a reusable scope handle that can also have a reusable cord, i.e. not necessarily cordless. In this system the front scope needle 20 with the shaft-retaining piece is disposable yet the rest of the handle is reusable. Even though the handle may be WiFi enabled it may still need a cord 231 to connect to a system, and the cord may not necessarily be sterile during the procedure. This embodiment includes a continuous bag or flexible material 230 interrupted by bands of plastic or snapping pieces to two or more of the areas of the handle, as illustrated for example at 232 and 234. The disposable bag is integrated with the disposable front needle and mounting piece of the scope and initially is folded or rolled up before the disposable scope part is attached to the reusable handle, as indicated in FIG. 28B. Once attached the bag 230 is unfolded or unrolled over first the rotating portion 36 of the reusable handle and then over the proximal fixed portion. At that location there is another snapping fixture (or the band 234 can be moved back) that will hold in place that portion of the bag over the rotating piece and part of the fixed portions of the handle. Then the rest of the bag is unfolded to cover the rest of the handle back and also the cable 231 all the way to a connection with the image processor 11 or monitor 12, which are non-sterile systems for data acquisition and/or control of the scope. The portion of the sterile barrier material 230 that is over the rotating part of then handle is designed so that adequate excess material is provided to allow the rotation of the two parts with respect to each other without constraint by the sterile barrier material or bag 230. This is also accomplished by means of gathering or folding the material or keeping loose material adequate to make multiple rotations. The material is primarily a cylindrical tube shape or similar, but it can be shaped as desired. In that sense the inner diameter of the disposable tube is as important a consideration as is the length of the stretched tube, as both define the limits of rotation of the fixed and rotating portion of the handle with respect to each other. If the diameter is as tight as the handle and wraps around tight without any extra slack in length, and the material is not stretchable, then rotation is hindered. If the diameter is very large, even with the tube of small length, then rotation is possible up to a specific angle without the material being stretched. The disposal of the bag and retainer rings or snaps or other fixtures at the one, two or more locations on the handle can be done by removing the bag via rolling from the cord end to the handle and then removing the disposable tip. Alternatively a separate ring with a disposable sterile barrier is inserted over the needle of the scope (even if all other parts of the scope are reusable) and then rolled and snapped as described above. During disposal this ring is removed first or last and the bag is rolled over the reusable scope.

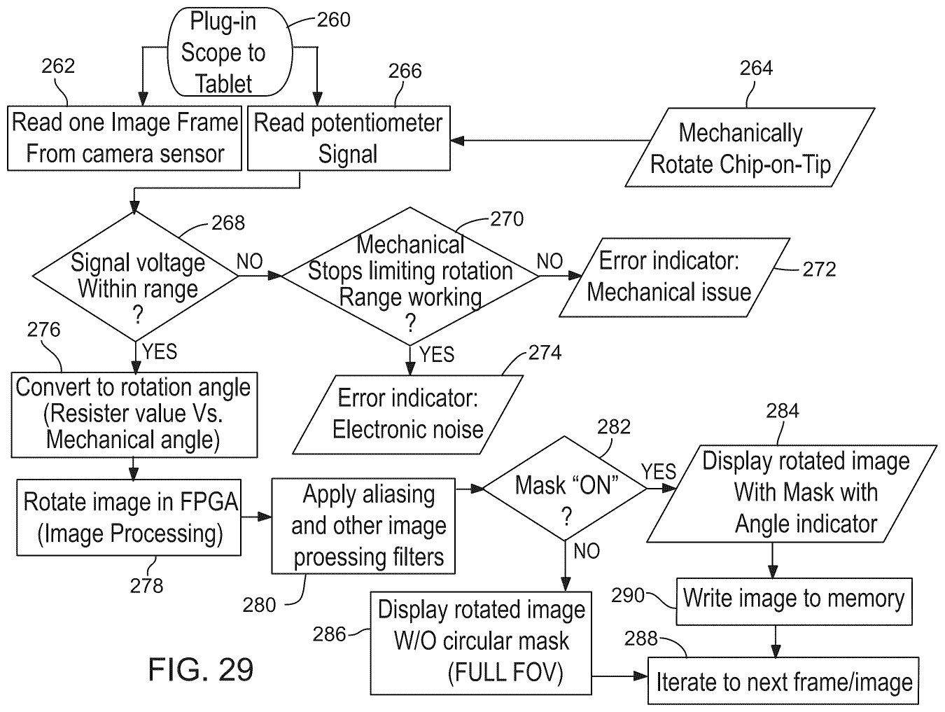

FIG. 29 is a flow chart indicating routine for reorientation of video images during rotation of the shaft/needle 20 with the side view scope of the invention. Per the block 260, the angle-view scope of the invention is plugged into a tablet or other monitor, indicated at 11 and 12 in the schematic view of FIG. 1, the image processor being shown at 11. As at 262 the programming reads an image frame from the camera sensor. This is of course a rapidly repeating process.

At 264 is indicated the rotation by a surgeon or technician of the chip-on-tip instrument shaft (with optics and camera) during a procedure. The potentiometer (or magnetometer) signal, produced as described above, is read by the programming as noted at the block 266. At the decision block 268 the system verifies that signal voltage from the potentiometer is within range, i.e. within a range that will indicate a valid angle of rotation. If not, a further decision block 270 checks for operation of mechanical stops. In either event errors are indicated, as noted in the blocks 272 and 274.

Assuming the signal voltage from the potentiometer is within range, the voltage signal is converted to a rotation angle as noted in the block 276. At the block 278, the image frame data is received, as is the rotation angle from the block 276. In response to the data from the block 276, the image is rotated in FPGA (field-programmable gate array), this occurring in the image processing program. The image rotation can also be done in other appropriate hardware such as a system-on-module with FPGA and/or CPU (computational processing unit) processor(s). At the block 280, aliasing and other image processing filters are applied. A circular mask could be applied in software (block 282) so that it displays as a round ring tangential to the sides of the rectangular image created by the rectangular optical sensor (CMOS, chip on tip). An indicator that specifies the deviation of angle from a reference position is also shown on the circular mask, as indicated in the block 284, and is able to shift around the perimeter of the mask so as to indicate the angle of the scope tip with respect to a reference angle. In most cases the reference angle is calibrated to match the horizontal or vertical of a patient orthonormal system, so as to give the doctor a real-time feedback of where the chip is truly located even if the image on the display is always referenced and corrected for then patient position and anatomy. This feature of masking an area of the rectangular image to show only a circular image is optional. The rotation angle indicator could also be optional or can also be applied to the periphery of the rectangular image itself without invoking a circular mask, as at the block 286. This gives the advantage of larger field of view. Saving videos or images in either a rotated or pure format is an option of the system, as noted in the blocks 288 and 290. This has implication in firmware choices of where the rotation takes place and how it is displayed.

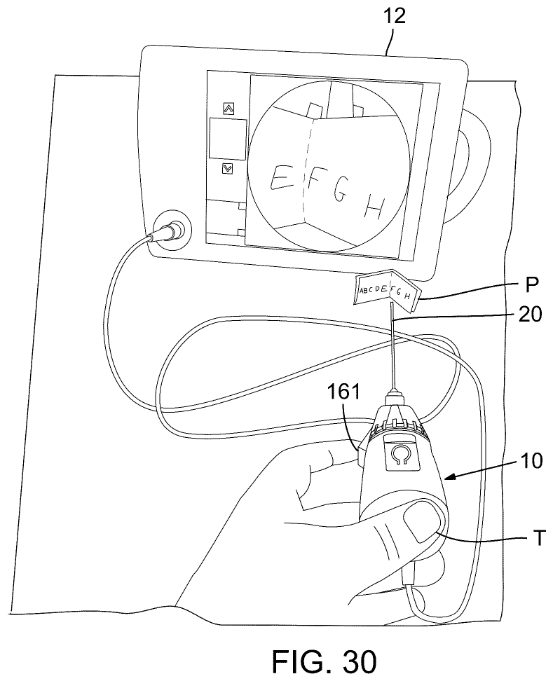

FIGS. 30, 31 and 32 show a simple demonstration of the image rotation correction feature of the invention, as well as illustrating the one-hand operation of the scope due to its construction. In these figures the scope 10 of the invention is held in a user's hand, with the shaft shown near a piece of note paper P, bearing letters A through H as an example. The user holds the body 16 of the handle between several fingers and the thumb T, while the index and middle fingers (in this particular example) engage a rotation shark fin 161, such as discussed earlier. The fin 161 is an grippable projection that can be located by tactile sensing and which is ergonomically positioned to enable one handed operation of the scope, for simultaneously holding the scope and rotating the needle and its side view.

In FIG. 30 the user holds the needle with camera and optics, in a position such that the right side of the range of letters is displayed EFGH. The image is shown on the screen 12, which can be a tablet computer. The scope needle 20 with its 30° viewing angle has been rotated clockwise, viewing to the right on the paper P. The illumination, following the viewing angle, can be seen mostly to the right on the paper.

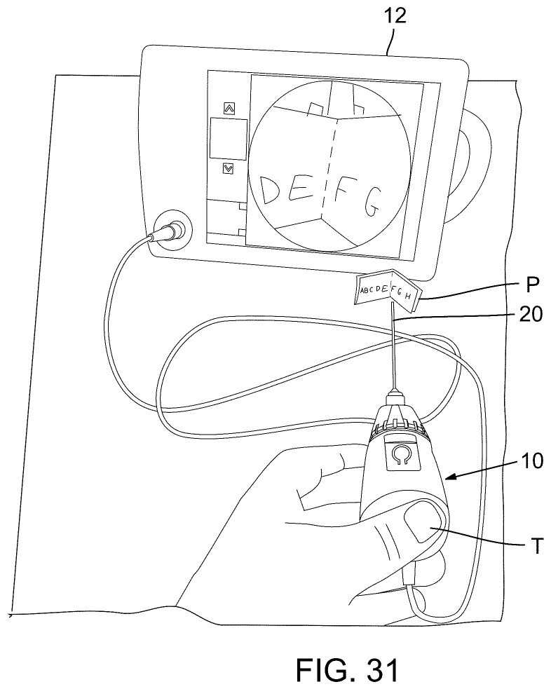

In the next view, FIG. 31, the user has rotated the scope tip counterclockwise, to approximately a middle position with the view centered but angled upwardly. The illumination has moved to about center of the scrap of paper P, and the image shows DEFG. This rotation of the angled scope will also lower the imaged letters to a lower position on the monitor, but here it is assumed the user is also able to adjust the pitch of the needle shaft 20 so as to keep the image of the letters essentially at mid-height of the monitor. As shown on the monitor, the image of the letters remains in proper orientation, the same orientation as that of FIG. 30, to provide continuity of viewing orientation for the user. The image processor work continuously to effect this correction.

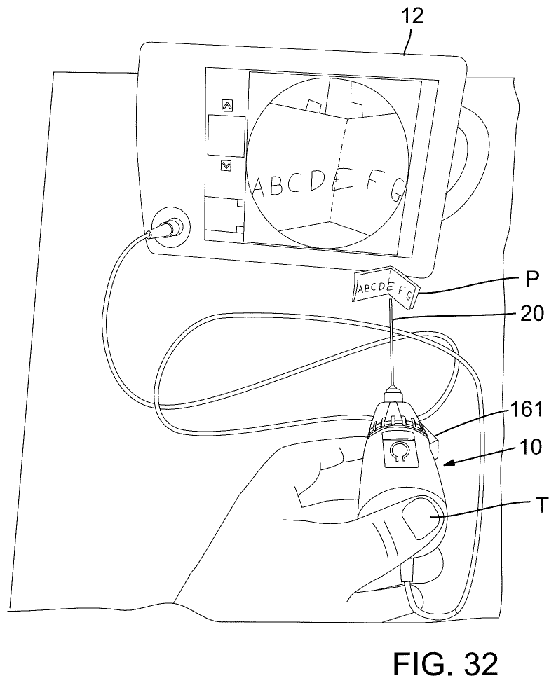

In FIG. 32 the user has rotated the tip of the scope further in the counterclockwise direction, moving the image farther left and revealing more of the letters at the beginning of the sequence. The illumination has moved left on the paper P. Again, the image of the letters is corrected in orientation to be consistently displayed.

The rotation of the scope tip effectively provides a much wider angle of view in all directions left, right, up and down. In the example shown in FIGS. 30 to 32 the instrument has been manipulated in pitch so as to keep the imaged letters proximal at mid-height, but if the scope is constrained by anatomy the pitch adjustment might not be possible, and in addition, the user will often desire to examine more tissue up and down, as well as left and right, so that features of tissue will change in height position with scope rotation.

From these views the configuration and size of the scope 10 relative to a person's hand are understood, as well as the ease of use in rotating the needle shaft (with angle viewing) using a finger or two. Rotation is easy, well-controlled and comfortable while the body of the scope is held in the palm or between the thumb and smaller fingers.

As explained above, the miniature videoscope of the invention is ergonomically designed to be operated and rotated with a single hand of the user. The configuration is such that the body of the device is held in the palm, or between the last two fingers on the palm, and the rotation fin near the distal end of the handle can easily be operated with one or two fingers.

Single hand operation of a device is known in the art. One example is handgun operation where ergonomics of holding the pistol grip within the palm while operating the trigger with a finger leads to dimensional constrains relevant to an average human hand. These principles are applied in the current invention. Configuration and dimensions of the scope of the invention are uniquely adapted to single hand operation.

Exemplary dimensions for the instrument are as follows:
Total length of handle (nose piece to cord): 12 cm (range of about 10 to 14 cm).
Distance from palm grip area to fin tip: 5 to 7 cm (range of about 4 to 8 cm, or 2 to 10 cm).
Location of control button(s) center with respect to back surface of back cap: 5 cm (range can be 3-9 cm with the best range within 5-8 cm).
Location of button(s) with respect to fin tip: 2 cm (range of 1 cm-5 cm).
Largest width/diameter of body/back cap: 5 cm (range 3-8 cm).
Diameter of rotating piece: max 4 cm and min 2 cm. Range of max about 2-6 cm and range of min about 1-5 cm.
Fin protrusion height 3 mm (range of 2 mm-10 mm and possibly 5 mm-15 mm or even larger). Fin can be removable and magnetically held or clippable onto cone piece 36.
Fin angle: 30 deg, range of 25 deg to 60 deg with 30-45 deg optimal.
Weight of handle of scope: reasonable to hold and manipulate in a single hand, preferably about 4-7 oz. (113-198 g.).

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A medical endoscope system for visualization of a patient's interior tissues or cavity in real time, comprising:
an endoscope device having a needle with a distal end for insertion into tissue and a proximal end with a handle,
the distal end having an imaging sensor and imaging optic for producing digital video images, the imaging sensor being connected to the handle for transmission of digital image data to a connected image processor remote from the endoscope device,
the needle being rotatable along a longitudinal axis with respect to a base part of the handle,
a fluid delivery cannula fitted over the needle, with a luer port connected to the cannula for receiving a fluid and delivering the fluid through the cannula to exit the cannula essentially at the distal end of the needle, the luer port being rotatable about a longitudinal axis independently of the cannula and the needle,
the image processor including image correction means for maintaining an upright consistent image orientation of video images when displayed despite rotation of the needle, and
a video monitor connected to the image processor to display video images of the patient's tissue or cavity from the camera in real time, the video images being corrected for rotational orientation and displaying consistent image orientation during use of the endoscope device with rotation of the needle.

2. The medical endoscope system of claim 1, wherein the imaging sensor and imaging
optic are angled at an acute angle from the longitudinal axis so as to produce side view video images when the needle is rotated.

3. The medical endoscope system of claim 1, wherein the needle forms a part of a disposable component which includes a needle base permanently secured to a proximal end of the needle and releasably connectable to the handle.

4. The medical endoscope system of claim 3, wherein the handle includes a distal end piece which is rotatable with respect to a proximal body portion of the handle that forms a part of the base part, with a manually engageable radial projection on the rotatable piece for rotation of the needle during use of the endoscope system, the disposable component being releasably connectable to the distal end piece of the handle.

5. The medical endoscope system of claim 1, wherein the body comprises a distal end piece, a body proximal of the distal end piece and a back cap as a proximal component, the distal end piece being secured in a snap-together connection with the body and the body being secured in a snap-together connection with the back cap, without screws.

6. The medical endoscope of claim 5, wherein the distal end piece is a rotatable part of the handle.

7. The medical endoscope system of claim 1, wherein the imaging sensor is connected to the image processor by wireless connection.

8. The medical endoscope system of claim 1, wherein the distal end of the needle includes an illumination device comprising a distal end of an optical fiber carrying light from a light source proximal in the endoscope device.

9. The medical endoscope system of claim 8, wherein the light source is an LED positioned in a needle base secured to the needle.

10. The medical endoscope system of claim 9, including a heat sink in contact with the LED for drawing heat from the LED.

11. The medical endoscope system of claim 1, wherein the distal end of the needle includes an illumination device comprising one or more LEDs.

12. The medical endoscope system of claim 1, including a rotation transducer operable between the handle and the needle, the transducer comprising a potentiometer, magnetometer or encoder monitoring rotational position of the needle relative to the handle and producing a signal sent to the image correction means.

13. The medical endoscope of claim 12, wherein the potentiometer, magnetometer or encoder is located off-axis from the needle's rotation axis, driven by gearing from the needle's rotational axis.

14. A medical endoscope system for visualization of a patient's interior tissues or cavity in real time, comprising:
an endoscope device having a needle with a distal end for insertion into tissue and a proximal end with a handle,
the distal end having an imaging sensor and imaging optic for producing digital video images, the imaging sensor being connected for transmission of digital image data to a connected image processor,
the needle being rotatable along a longitudinal axis with respect to the handle, and including a needle base or scope cavity permanently secured to the needle, the needle base being rotatably connected to a fixed element, the fixed element being removably attached to the handle, and
a video monitor connected to the image processor to display video images of the patient's tissue or cavity from the image sensor in real time.

15. The medical endoscope system of claim 14, further including a radially extending lever on the needle base or scope cavity, positioned to be manually operated when the handle is held, to rotate the needle base and needle relative to the handle.

16. The medical endoscope system of claim 14, wherein the handle includes the image processor.

17. The medical endoscope system of claim 16, wherein the handle and the image processor include wireless connection means to communicate image data from the imaging sensor to the image processor, and the handle including a battery for supplying power to the fixed element, the needle base and the needle.

18. The medical endoscope system of claim 14, wherein the needle base or the fixed element includes electronics to receive the digital image data, with a wireless transmitter to send the data wirelessly to the image processor, and wherein the handle includes a battery supplying power to the needle and electronics when the fixed element is attached to the handle.

19. The medical endoscope system of claim 14, wherein the image processor includes image correction means for maintaining an upright consistent image orientation of video images when displayed despite rotation of the needle.

20. The medical endoscope system of claim 14, wherein the image correction means includes a rotation sensing transducer between the needle base and the fixed element.

21. The medical endoscope system of claim 14, further including a LED residing in the handle and optical fibers in the needle, the LED optically communicating with the optical fibers of the needle when the fixed element is connected to the handle.

22. The medical endoscope system of claim 14, wherein the needle includes illumination provided by at least one LED positioned in the distal tip of the needle.

23. The medical endoscope system of claim 14, further including an encapsulation device for sterile encapsulation of the reusable handle, the encapsulation device being connectable to the fixed element of the needle when the fixed element is connected to the handle.

24. The medical endoscope system of claim 14, in combination with a fluid delivery cannula fitted over the needle, with a luer port connected to the cannula for receiving a fluid and delivering the fluid through the cannula to exit the cannula essentially at the distal end of the needle, the luer port being rotatable about a longitudinal axis independently of the cannula and the needle.

25. The medical endoscope system of claim 1, in combination with a fluid delivery cannula fitted over the needle, with a luer port for receiving a fluid and delivering the fluid through the cannula to exit the cannula essentially at the distal end of the needle, the luer port being rotatable about a longitudinal axis independently of the cannula and the needle.

26. A medical endoscope system for visualization of a patient's interior tissues or cavity in real time, comprising:
an endoscope device having a needle with a distal end for insertion into tissue and a proximal end with a handle,
the distal end having a chip-on-tip imaging sensor and imaging optic for producing digital video images, the imaging sensor being connected for transmission of digital image data to an image processor remote from the endoscope device,
the needle being rotatable along a longitudinal axis with respect to a base part of the handle,
the handle including a rotatable distal end connected to the needle, rotatably connected to the base part of the handle and having a finger-engaging element for rotating the handle relative to the base part of the handle using a finger or thumb,
the handle being configured to be held and manipulated with a single hand with the base part contacted by a palm or thumb and one or two fingers or a thumb on the finger-engaging element, the base part having a diameter no greater than about 6 cm and the finger-engaging element being about 2 to 10 cm distal of a proximal end of the base part of the handle, and
a video monitor connected to the image processor to display video images of the patient's tissue or cavity from the imaging sensor in real time, the video images being corrected for rotational orientation and displaying consistent image orientation during use of the endoscope device with rotation of the needle.

27. The medical endoscope system of claim 26, wherein the image processor includes image correction means for maintaining an upright consistent image orientation of video images when displayed despite rotation of the needle.

28. The medical endoscope system of claim 26, wherein the imaging sensor and imaging optic are angled at an acute angle from the longitudinal axis so as to produce side view video images when the needle is rotated.

29. The medical endoscope system of claim 26, wherein the handle comprises a distal end piece, a main body piece proximal of the distal end piece and a back cap as a proximal component, the distal end piece being secured in a snap-together connection with the main body piece and the main body piece being secured in a snap-together connection with the back cap, without screws or other metal fasteners.

30. The medical endoscope system of claim 26, wherein the needle forms a part of a disposable component which includes a needle base permanently secured to a proximal end of the needle and releasably connectable to the handle.

31. The medical endoscope system of claim 30, wherein the handle includes a distal end piece which is rotatable with respect to a proximal body portion of the handle that forms a part of the base part, with a manually engageable radial projection on the rotatable piece for rotation of the needle during use of the endoscope system, the disposable component being releasably connectable to the distal end piece of the handle.

32. The medical endoscope system of claim 31, wherein the radial projection comprises a finger or thumb-engageable fin.

33. The medical endoscope system of claim 30, wherein the needle base is separable from the handle by manipulation of one or more levers or latches at the exterior of the handle.

34. The medical endoscope of claim 29, wherein the distal end piece is a rotatable part of the handle.

35. The medical endoscope system of claim 26, wherein the distal end of the needle includes an illumination device comprising a tip of an optical fiber carrying light from a light source proximal in the endoscope device.

36. The medical endoscope system of claim 35, wherein the light source is an LED positioned in a needle base secured to the needle.

37. The medical endoscope system of claim 36, including a heat sink in contact with the LED for drawing heat from the LED.

38. The medical endoscope system of claim 26, including a rotation transducer operable between the handle and the needle, the transducer comprising a potentiometer monitoring rotational position of the needle relative to the handle and producing a signal sent to the image correction means.

39. The medical endoscope system of claim 26, wherein the needle and a connected needle base are removable from the handle and disposable, and wherein the handle is reusable.

40. The medical endoscope system of claim 39, including a needle base or scope cavity permanently secured to the needle, the scope cavity being rotatably connected to a fixed element, the fixed element being removably connected non-rotatably to the base part of the handle.

41. The medical endoscope system of claim 26, wherein the needle forms part of a disposable component which includes a needle base permanently secured to a proximal end of the needle, the needle base being releasably connectable to the handle, and the endoscope device being wirelessly connected to the image processor, a battery being included in the handle, and further including a charging base adapted to rest on a surface and having features to receive a proximal end of the handle for charging the battery when the body is placed on the charging base.

42. A medical endoscope system for visualization of a patient's interior tissues or cavity in real time, comprising:
an endoscope device having a distal end with a needle for insertion into tissue and a proximal end with a handle,
the needle having a distal tip with an imaging sensor and imaging optic for producing digital video images, the imaging sensor being connected to the handle for transmission of digital image data to a connected image processor remote from the endoscope device,
the needle being rotatable along a longitudinal axis with respect to a base part of the handle,
the image processor including image correction means for maintaining an upright consistent image orientation of video images when displayed despite rotation of the needle,
a video monitor connected to the image processor to display video images of the patient's tissue or cavity from the imaging sensor in real time, the video images being corrected for rotational orientation and displaying consistent image orientation during use of the endoscope device with rotation of the needle,
the endoscope device being separable into a reuseable part comprising said base part of the handle, and a disposable part comprising the needle and a needle base fixed to a proximal end of the needle,
a proximal disposable shell surrounding and attached to the handle, for maintaining sterility of the handle during use, and
a distal disposable shell surrounding and attached to the needle base, with attachment means for snapping together the proximal and distal disposable shells to form a complete shell while coupling the needle base to the handle mechanically and electrically, and with means for effecting rotation of the needle base and needle from exterior of the complete shell during use of the endoscope device.

43. The medical endoscope system of claim 42, wherein the means for effecting rotation comprises a rotatable connection between the distal and proximal disposable shells when the shells are snapped together such that rotation of the distal disposable shell is effective to rotate the needle.

44. The medical endoscope of claim 42, in combination with a charging station, the handle being connectable to the image processor wirelessly and the handle including a rechargeable battery, the handle being removable from the proximal disposable shell so as to be received in the charging station for charging, and the distal disposable shell, affixed to the needle and needle base, being securable to the handle by snap-together connection without need for manual contact with the handle while on the charging station, whereby once the distal disposable shell with needle and needle base are secured to the handle, the handle can be inserted into the proximal disposable shell after which the proximal end and distal disposable shells can be snapped together.
